(19) United States
(12) Patent Application Publication
(10) Pub. No.: US 2022/0288015 A1
(43) Pub. Date: Sep. 15, 2022

(54) CANNABINOID ACID ESTER COMPOSITIONS AND USES THEREOF

(71) Applicant: EPM GROUP, INC., San Francisco, CA (US)

(72) Inventors: Reshef Swisa, San Francisco, CA (US); Salomon Sacal, San Francisco, CA (US); Ron Shahrabani, San Francisco, CA (US)

(21) Appl. No.: 17/637,031

(22) PCT Filed: Jun. 19, 2020
(86) PCT No.: PCT/US2020/038748

§ 371 Date: Feb. 21, 2022

Related U.S. Application Data
(60) Provisional application No. 62/890,081, filed on Aug. 22, 2019.
(60) Provisional application No. 62/890,079, filed on Aug. 22, 2019.
(60) Provisional application No. 62/890,080, filed on Aug. 22, 2019.
(60) Provisional application No. 62/890,085, filed on Aug. 22, 2019.
(60) Provisional application No. 62/890,089, filed on Aug. 22, 2019.
(60) Provisional application No. 62/890,090, filed on Aug. 22, 2019.
(60) Provisional application No. 62/963,044, filed on Jan. 19, 2020.
(60) Provisional application No. 62/963,041, filed on Jan. 19, 2020.
(60) Provisional application No. 62/963,043, filed on Jan. 19, 2020.

(51) Int. Cl. A61K 31/352; A61K 31/05; A61K 36/185; A61K 45/06

(52) U.S. Cl. CPC A61K 31/352; A61K 31/05; A61K 36/185; A61K 45/06

(57) ABSTRACT

The present disclosure provides pharmaceutical compositions including a cannabinoid acid ester compound alone or in combination with one or more additional cannabinoid compounds. In some embodiments, the cannabinoid acid ester compound is a tetrahydrocannabinolic acid (THCA) ester. In some embodiments, the cannabinoid acid ester compound is a cannabigerolic (CBGA) acid ester. In some embodiments, the cannabinoid acid ester compound is a cannabinolic (CBNA) acid ester. A variety of therapeutic applications in which the cannabinoid acid ester compounds and pharmaceutical compositions find use are also provided, including combination therapies using cannabinoid acid ester compounds and one or more additional therapeutic agents.

### CROSS-REFERENCE TO RELATED APPLICATIONS

This application claims the benefit of U.S. Provisional Application No. 62/890,079 filed Aug. 22, 2019; 62/890,080 filed Aug. 22, 2019; 62/890,081 filed Aug. 22, 2019; 62/890,085 filed Aug. 22, 2019; 62/890,089 filed Aug. 22, 2019; 62/890,090 filed Aug. 22, 2019; 62/963,044 filed Jan. 19, 2020; 62/963,041 filed Jan. 19, 2020; and 62/963,043 filed Jan. 19, 2020 which applications are hereby incorporated by reference in their entirety.

### INTRODUCTION

Tetrahydrocannabinol (THC) is the principal psychotropic phytocannabinoid compound present in the plant Cannabis sativa and constitutes up to 25% of the cannabinoids in Cannabis. On the other hand, cannabigerol (CBG) and cannabinol (CBN) are minor cannabinoids found only in trace amounts in Cannabis. Both THC and CBN can act as partial agonists at the CB1 receptor with higher affinity to CB2 receptors, with CBN having lower affinity at CB2 relative to THC. The two main cannabinoids in the plant are cannabidiol (CBD) and THC.

Another constituents of Cannabis are the cannabinoid acids, such as tetrahydrocannabinolic acid (THCA), cannabigerolic acid (CBGA), and cannabinolic acid (CBNA), which are the precursors of the natural cannabinoids. For example, THC is produced in the plant by decarboxylation of THCA. CBGA is also the precursor of the natural cannabinoid acids. Due to their relative instabilities to decarboxylation, the chemical structures of THCA, CBGA, and CBNA were first elucidated by analysis of their ester derivatives, namely tetrahydrocannabinolic acid methyl ester (THCA-Me), cannabigerolic acid methyl ester (CBGA-Me), and cannabinolic acid methyl ester (CBNA-Me).

The cannabinoid-based compounds may have a variety of biological activities and effects. Elucidation of desirable non-traditional biological activities of cannabinoid acid compounds, and the demonstration of useful therapeutic applications for such compounds is of interest.

### SUMMARY

The present disclosure provides pharmaceutical compositions including a cannabinoid acid ester compound alone or in combination with one or more additional cannabinoid compounds. In some embodiments, the cannabinoid acid ester compound is a tetrahydrocannabinolic acid (THCA) ester. In some embodiments, the cannabinoid acid ester compound is a cannabigerolic acid (CBGA) ester. In some embodiments, the cannabinoid acid ester compound is a cannabinolic acid (CBNA) ester. A variety of therapeutic applications in which the cannabinoid acid ester compounds and pharmaceutical compositions find use are also provided, including combination therapies using cannabinoid acid ester compounds and one or more additional therapeutic agents.

### DETAILED DESCRIPTION

### Cannabinoid Acid Ester Compounds

The term “cannabinoid” refers to a compound that interacts with and binds to a cannabinoid receptor. Binding compounds (i.e., ligands) for cannabinoid receptors include, but are not limited to, endocannabinoids (produced naturally in the body by human and animals), phytocannabinoids (found in Cannabis and some other plants), and synthetic cannabinoids (manufactured artificially). The term “cannabinoid acid” refers to a carboxylic acid-substituted form or derivative of a cannabinoid compound. The term “cannabinoid acid ester” refers to an ester form of a cannabinoid acid compound, where the carboxylic acid substituent of the parent cannabinoid acid is in an ester form.

The term “tetrahydrocannabinol” refers to the phytocannabinoid tetrahydrocannabinol (THC).

In some embodiments, THC can be referred to as 6,6,9-trimethyl-3-pentyl-6a,7,8,10a-tetrahydro-6H-benzo[c]chromen-1-ol, or Δ⁹-tetrahydrocannabinol (THC), and has the following structure:

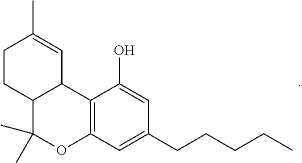 [SMILES: CCCCCc1cc(O)c2c(c1)OC(C)(C)C1CCC(C)=CC21]

In some embodiments, THC can be referred to as 6,6,9-trimethyl-3-pentyl-6a,7,10,10a-tetrahydro-6H-benzo[c]chromen-1-ol, or Δ⁸-tetrahydrocannabinol (THC), and has the following structure:

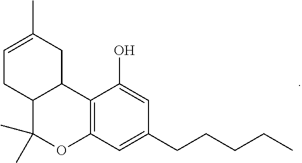 [SMILES: CCCCCc1cc(O)c2c(c1)OC(C)(C)C1CC=C(C)CC21]

THC can be obtained from industrial hemp extract or from Cannabis extract. Tetrahydrocannabinol may be obtained from plant extract or may be prepared synthetically (manufactured artificially).

The term “tetrahydrocannabinolic acid” (THCA) refers to a carboxylic acid form of THC, e.g., a 2-carboxy derivative of a THC compound.

In some embodiments, Δ⁹-THCA can be referred to as 1-hydroxy-6,6,9-trimethyl-3-pentyl-6a,7,8,10a-tetrahydro-6H-benzo[c]chromene-2-carboxylic acid and has the following structure:

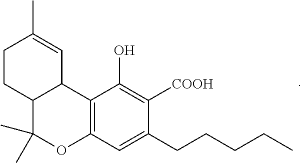 [SMILES: CCCCCc1cc2c(c(O)c1C(=O)O)C1C=C(C)CCC1C(C)(C)O2]

In some embodiments, Δ⁸-THCA can be referred to as 1-hydroxy-6,6,9-trimethyl-3-pentyl-6a,7,10,10a-tetrahydro-6H-benzo[c]chromene-2-carboxylic acid and has the following structure:

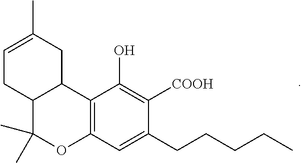 [SMILES: CCCCCc1cc2c(c(O)c1C(=O)O)C1CC(C)=CCC1C(C)(C)O2]

The terms “tetrahydrocannabinolic acid ester” and “tetrahydrocannabinolic ester” are used interchangeably and refer to esters of THCA. The term “THCA-Me” refers to tetrahydrocannabinolic acid methyl ester, the methyl ester form of THCA.

The term “cannabigerol” refers to the phytocannabinoid cannabigerol (CBG). CBG can be referred to as (E)-2-(3,7-dimethylocta-2,6-dien-1-yl)-5-pentylbenzene-1,3-diol, and has the following structure:

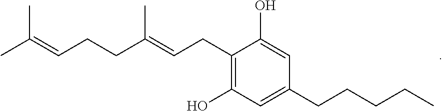 [SMILES: CCCCCc1cc(O)c(C/C=C(\C)CCC=C(C)C)c(O)c1]

CBG can be obtained from industrial hemp extract (with, in some cases, a trace amount of THC) or from Cannabis extract. Cannabigerol may be obtained from plant extract or may be prepared synthetically (manufactured artificially).

Cannabigerolic acid (CBGA) is an acid form of CBG. CBGA can be referred to a (E)-3-(3,7-dimethylocta-2,6-dien-1-yl)-2,4-dihydroxy-6-pentylbenzoic acid and has the following structure:

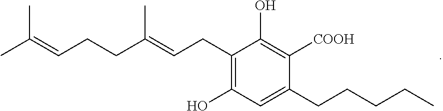 [SMILES: CCCCCc1cc(O)c(C/C=C(\C)CCC=C(C)C)c(O)c1C(=O)O]

The terms “cannabigerolic acid ester” and “cannabigerolic ester” are used interchangeably and refer to esters of CBGA. The term “CBGA-Me” refers to cannabigerolic acid methyl ester, the methyl ester form of CBGA.

The term “cannabinol” refers to the phytocannabinoid cannabinol (CBN). CBN can be referred to as 6,6,9-trimethyl-3-pentyl-6H-benzo[c]chromen-1-ol, and has the following structure:

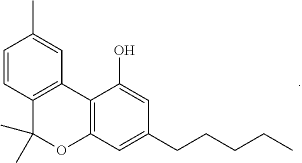 [SMILES: CCCCCc1cc(O)c2c(c1)OC(C)(C)c1ccc(C)cc1-2]

CBN can be obtained from industrial hemp extract (with, in some cases, a trace amount of THC) or from Cannabis extract. Cannabinol may be obtained from plant extract or may be prepared synthetically (manufactured artificially).

Cannabinolic acid (CBNA) is an acid form of CBN. CBNA can be referred to as 1-hydroxy-6,6,9-trimethyl-3-pentyl-6H-benzo[c]chromene-2-carboxylic acid and has the following structure:

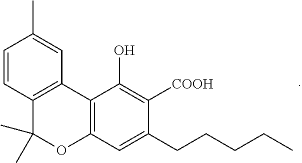 [SMILES: CCCCCc1cc2c(c(O)c1C(=O)O)-c1cc(C)ccc1C(C)(C)O2]

The terms “cannabinolic acid ester” and “cannabinolic ester” are used interchangeably and refer to esters of CBNA. The term “CBNA-Me” refers to cannabinolic acid methyl ester, the methyl ester form of CBNA.

The present disclosure provides compositions including cannabinoid acid ester compounds that find use in a variety of therapeutic applications, e.g., as described herein. The inventors have discovered and elucidated a variety of desirable biological activities and new therapeutic applications of cannabinoid acid ester compounds, e.g., as described herein. The ester form of a cannabinoid acid compound can be a desirable and useful form of a bioactive compound suitable for administration to a subject in a therapeutic application (e.g., as described herein). In some cases, the ester form is generally more stable, e.g., in vivo, than the parent cannabinoid acid compound.

In some embodiments, the cannabinoid acid ester compound is a tetrahydrocannabinolic acid ester (THCA ester).

In some embodiments, the cannabinoid acid ester compound is a cannabigerolic acid ester (CBGA ester).

In some embodiments, the cannabinoid acid ester compound is a cannabinolic acid ester (CBNA ester).

A variety of cannabinoid acid compounds can be adapted for use in the therapeutic methods described herein in an ester form. Any convenient ester forms of cannabinoid acid compounds can be utilized. The particular ester form of the cannabinoid acid compound can be selected to provide for a desired stability profile and half-lives in vivo of the administered drug and any active metabolites thereof.

As described herein, cannabinoid acid compounds of interest include tetrahydrocannabinolic acid (THCA), analogs, and isomers of THCA that have the same core structure but which include different substituents, e.g., as a replacement for the pentyl group of THCA. In some embodiments, the cannabinoid acid ester compound is a THCA ester or analog thereof of formula (I):

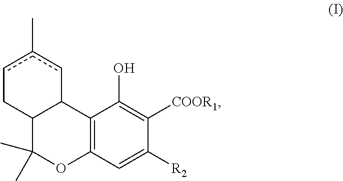

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof.

In some embodiments, the THCA ester compound of formula (I) is a THCA ester compound of formula (Ia):

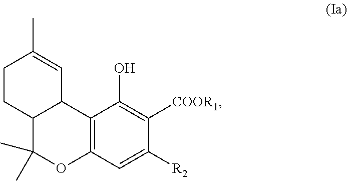

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof.

In some embodiments, the THCA ester compound of formula (I) is a THCA ester compound of formula (Ib):

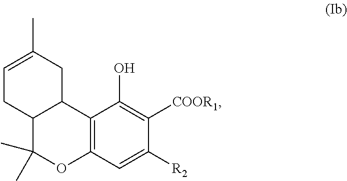

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof.

In some embodiments, the THCA ester compound of formula (I) is the Δ⁹-tetrahydrocannabinolic acid methyl ester (Δ⁹-THCA-Me) compound of formula (Ic):

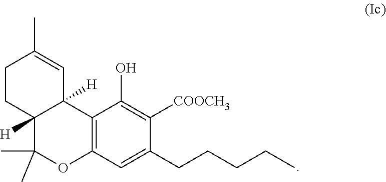 [SMILES: CCCCCc1cc2c(c(O)c1C(=O)OC)[C@@H]1C=C(C)CC[C@H]1C(C)(C)O2]

In some embodiments, the THCA ester compound of formula (I) is the Δ⁸-tetrahydrocannabinolic acid methyl ester (Δ⁸-THCA-Me) compound of formula (Id):

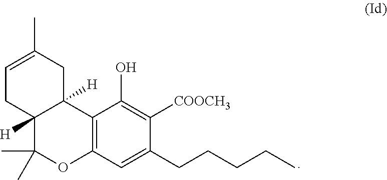 [SMILES: CCCCCc1cc2c(c(O)c1C(=O)OC)[C@@H]1CC(C)=CC[C@H]1C(C)(C)O2]

As described herein, cannabinoid acid compounds of interest include cannabigerolic acid (CBGA), analogs, and isomers of CBGA that have the same core structure but which include different substituents, e.g., as a replacement for the pentyl group of CBGA. In some embodiments, the cannabinoid acid ester compound is a CBGA ester or analog thereof of formula (II):

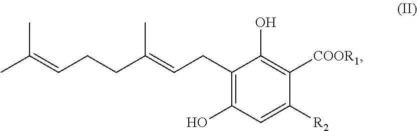

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof.

In some embodiments, the CBGA ester compound of formula (II) is a CBGA ester compound of formula (IIa):

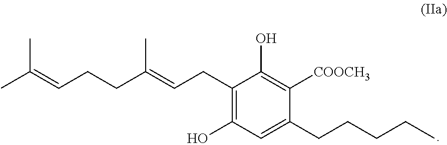 [SMILES: CCCCCc1cc(O)c(C/C=C(\C)CCC=C(C)C)c(O)c1C(=O)OC]

As described herein, cannabinoid acid compounds of interest include cannabinolic acid (CBNA), analogs, and isomers of CBNA that have the same core structure but which include different substituents, e.g., as a replacement for the pentyl group of CBNA. In some embodiments, the cannabinoid acid ester compound is a CBNA ester or analog thereof of formula (III):

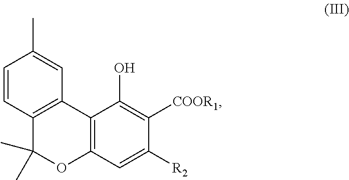

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof.

In some embodiments, the CBNA ester compound of formula (III) is a CBNA ester compound of formula (IIIa):

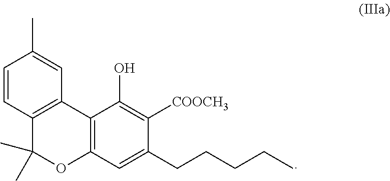 [SMILES: CCCCCc1cc2c(c(O)c1C(=O)OC)-c1cc(C)ccc1C(C)(C)O2]

Also provided are deuterated analogs of cannabinoid acid ester compounds.

In some cases, the compounds are deuterated analogs of THCA ester compounds. A deuterated analog of a compound of formulae (I)-(Id) is a compound where one or more hydrogen atoms are substituted with a deuterium. In some embodiments, the deuterated analog is a compound of formula (I) that includes a deuterated R₁ group. In some embodiments of a deuterated analog of formula (I), R₁ is a C₁-C₁₀ alkyl or substituted C₁-C₁₀ alkyl including at least one deuterium atom. In some embodiments of a deuterated analog of formula (I), R₁ is a C₁-C₆ alkyl or substituted C₁-C₆ alkyl including at least one deuterium atom. In some embodiments of a deuterated analog of formula (I), R₁ is —CD₃. In some embodiments of a deuterated analog of formula (I), R₁ is —CD₂-CD₃. In some embodiments of a deuterated analog of formula (I), the deuterated analog of the cannabinoid acid ester compound includes an R₂ group that is deuterated. In some embodiments of a deuterated analog of formula (I), R₂ is a C₁-C₁₀ alkyl or substituted C₁-C₁₀ alkyl including at least one deuterium atom. In some embodiments of a deuterated analog of formula (I), R₂ is a C₂-C₆ alkyl or substituted C₂-C₆ alkyl including at least one deuterium atom.

In some embodiments, the compound is a deuterated analog of THCA-Me ester of formula (IV):

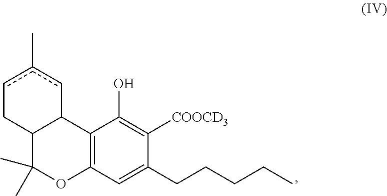

or a pharmaceutically acceptable salt thereof.

In some embodiments of formula (IV), the deuterated analog of THCA-Me ester is the compound of formula (IVa):

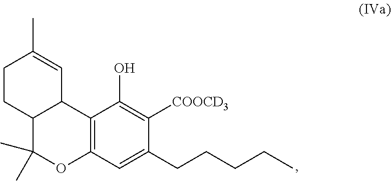

or a pharmaceutically acceptable salt thereof.

In some embodiments of formula (IV), the deuterated analog of THCA-Me is the compound of formula (IVb):

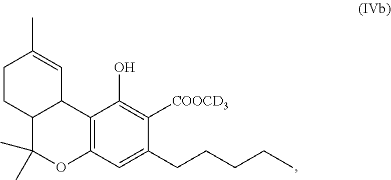

or a pharmaceutically acceptable salt thereof.

In some embodiments of formula (IV), the deuterated analog of THCA-Me is the compound of formula (IVc):

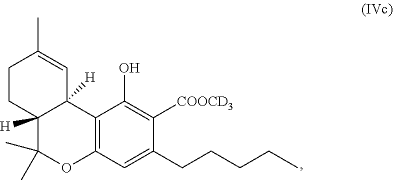

or a pharmaceutically acceptable salt thereof.

In some embodiments of formula (IV), the deuterated analog of THCA-Me is the compound of formula (IVd):

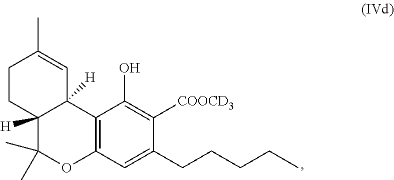

or a pharmaceutically acceptable salt thereof.

In some cases, the compounds are deuterated analogs of CBGA ester compounds. A deuterated analog of a compound of formulae (II) and (IIa) is a compound where one or more hydrogen atoms are substituted with a deuterium. In some embodiments, the deuterated analog is a compound of formula (II) that includes a deuterated R₁ group. In some embodiments of a deuterated analog of formula (II), R₁ is a C₁-C₁₀ alkyl or substituted C₁-C₁₀ alkyl including at least one deuterium atom. In some embodiments of a deuterated analog of formula (II), R₁ is a C₁-C₆ alkyl or substituted C₁-C₆ alkyl including at least one deuterium atom. In some embodiments of a deuterated analog of formula (II), R₁ is —CD₃. In some embodiments of a deuterated analog of formula (II), R₁ is —CD₂-CD₃. In some embodiments of a deuterated analog of formula (II), the deuterated analog of the cannabinoid acid ester compound includes an R₂ group that is deuterated. In some embodiments of a deuterated analog of formula (II), R₂ is a C₁-C₁₀ alkyl or substituted C₁-C₁₀ alkyl including at least one deuterium atom. In some embodiments of a deuterated analog of formula (II), R₂ is a C₂-C₆ alkyl or substituted C₂-C₆ alkyl including at least one deuterium atom.

In some embodiments, the compound is a deuterated analog of CBGA-Me ester of formula (V):

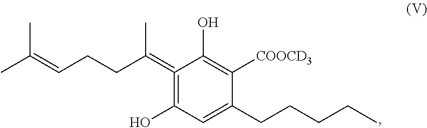

or a pharmaceutically acceptable salt thereof.

In some cases, the compounds are deuterated analogs of CBNA ester compounds. A deuterated analog of a compound of formulae (III) or (IIIa) is a compound where one or more hydrogen atoms are substituted with a deuterium. In some embodiments, the deuterated analog is a compound of formula (III) that includes a deuterated R₁ group. In some embodiments of a deuterated analog of formula (III), R₁ is a C₁-C₁₀ alkyl or substituted C₁-C₁₀ alkyl including at least one deuterium atom. In some embodiments of a deuterated analog of formula (III), R₁ is a C₁-C₆ alkyl or substituted C₁-C₆ alkyl including at least one deuterium atom. In some embodiments of a deuterated analog of formula (II), R₁ is —CD₃. In some embodiments of a deuterated analog of formula (III), R₁ is —CD₂-CD₃. In some embodiments of a deuterated analog of formula (III), the deuterated analog of the cannabinoid acid ester compound includes an R₂ group that is deuterated. In some embodiments of a deuterated analog of formula (III), R₂ is a C₁-C₁₀ alkyl or substituted C₁-C₁₀ alkyl including at least one deuterium atom. In some embodiments of a deuterated analog of formula (III), R₂ is a C₂-C₆ alkyl or substituted C₂-C₆ alkyl including at least one deuterium atom.

In some embodiments, the compound is a deuterated analog of CBNA-Me ester of formula (VI):

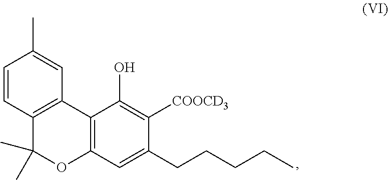

or a pharmaceutically acceptable salt thereof.

Also provided are fluorinated analogs of cannabinoid acid ester compounds.

In some cases, the compounds are fluorinated analogs of THCA ester compounds. A fluorinated analog of a compound of formulae (I)-(Id) is a compound where one or more hydrogen atoms are substituted with a fluorine. In some embodiments of the compound of formula (I), the fluorinated analog includes an R₁ group that is fluorinated. In some embodiments of formula (I), R₁ is a substituted C₁-C₁₀ alkyl including at least one fluoro substituent. In some embodiments of formula (I), R₁ is a substituted C₁-C₆ alkyl including at least one fluoro substituent. In some embodiments of formula (I), R₁ is —CF₃. In some embodiments of formula (I), R₁ is —CF₂—CF₃. In some embodiments of the compound of formula (I), the fluorinated analog of the cannabinoid acid ester compound includes an R₂ group that is fluorinated. In some embodiments of formula (I), R₂ is a substituted C₁-C₁₀ alkyl including at least one fluoro substituent. In some embodiments of formula (I), R₂ is a substituted C₂-C₆ alkyl including at least one fluoro substituent.

In some embodiments of the compound of formula I, the compound is a fluorinated analog of THCA ester. In another embodiment, the fluorinated analog of THCA ester is a compound of formula (VII):

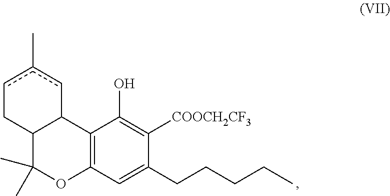

or a pharmaceutically acceptable salt thereof.

In some embodiments of the compound of formula (VII), the fluorinated analog of THCA ester is the compound of formula (VIIa):

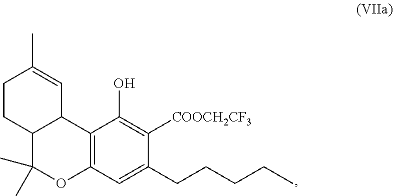

or a pharmaceutically acceptable salt thereof.

In some embodiments of the compound of formula (VII), the fluorinated analog of THCA ester is the compound of formula (VIIb):

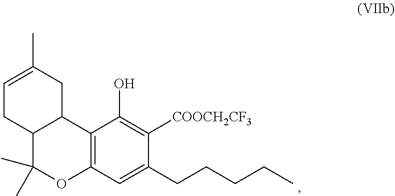

or a pharmaceutically acceptable salt thereof.

In some embodiments of the compound of formula (VII), the fluorinated analog of THCA ester is the compound of formula (VIIc):

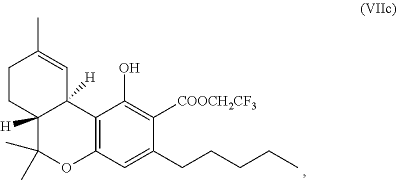

or a pharmaceutically acceptable salt thereof.

In some embodiments of the compound of formula (VII), the fluorinated analog of THCA ester is the compound of formula (VIId):

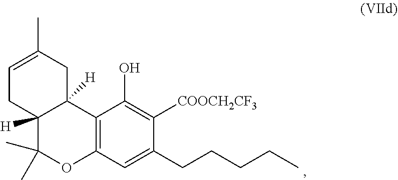

or a pharmaceutically acceptable salt thereof.

In some cases, the compounds are fluorinated analogs of CBGA ester compounds. A fluorinated analog of a compound of formulae (II) or (IIa) is a compound where one or more hydrogen atoms are substituted with a fluorine. In some embodiments of the compound of formula (II), the fluorinated analog includes an R₁ group that is fluorinated. In some embodiments of formula (II), R₁ is a substituted C₁-C₁₀ alkyl including at least one fluoro substituent. In some embodiments of formula (II), R₁ is a substituted C₁-C₆ alkyl including at least one fluoro substituent. In some embodiments of formula (II), R₁ is —CF₃. In some embodiments of formula (II), R₁ is —CF₂—CF₃. In some embodiments of the compound of formula (II), the fluorinated analog of the cannabinoid acid ester compound includes an R₂ group that is fluorinated. In some embodiments of formula (II), R₂ is a substituted C₁-C₁₀ alkyl including at least one fluoro substituent. In some embodiments of formula (II), R₂ is a substituted C₂-C₆ alkyl including at least one fluoro substituent.

In some embodiments of the compound of formula II, the compound is a fluorinated analog of CBGA ester. In another embodiment, the fluorinated analog of CBGA ester is a compound of formula (VIII):

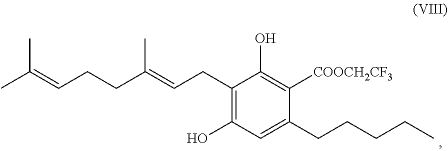

or a pharmaceutically acceptable salt thereof.

In some cases, the compounds are fluorinated analogs of CBNA ester compounds. A fluorinated analog of a compound of formulae (III) or (IIIa) is a compound where one or more hydrogen atoms are substituted with a fluorine. In some embodiments of the compound of formula (III), the fluorinated analog includes an R₁ group that is fluorinated. In some embodiments of formula (III), R₁ is a substituted C₁-C₁₀ alkyl including at least one fluoro substituent. In some embodiments of formula (III), R₁ is a substituted C₁-C₆ alkyl including at least one fluoro substituent. In some embodiments of formula (III), R₁ is —CF₃. In some embodiments of formula (III), R₁ is —CF₂—CF₃. In some embodiments of the compound of formula (III), the fluorinated analog of the cannabinoid acid ester compound includes an R₂ group that is fluorinated. In some embodiments of formula (III), R₂ is a substituted C₁-C₁₀ alkyl including at least one fluoro substituent. In some embodiments of formula (III), R₂ is a substituted C₂-C₆ alkyl including at least one fluoro substituent.

In some embodiments of the compound of formula III, the compound is a fluorinated analog of CBNA ester. In another embodiment, the fluorinated analog of CBNA ester is a compound of formula (IX):

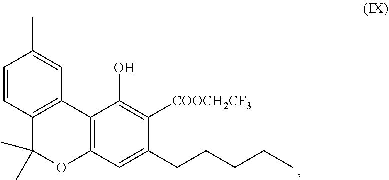

or a pharmaceutically acceptable salt thereof.

In some embodiments of formula (I), the compound is Δ⁹-tetrahydrocannabinolic acid methyl ester (Δ⁹-THCA-Me) compound of formula (Ic):

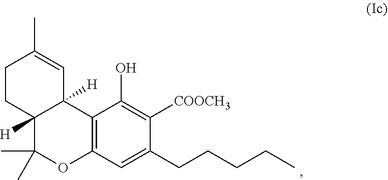 [SMILES: CCCCCc1cc2c(c(O)c1C(=O)OC)[C@@H]1C=C(C)CC[C@H]1C(C)(C)O2]

or a pharmaceutically acceptable salt thereof.

In some embodiments of formula (I), the compound is Δ⁸-tetrahydrocannabinolic acid methyl ester (Δ⁸-THCA-Me) compound of formula (Id):

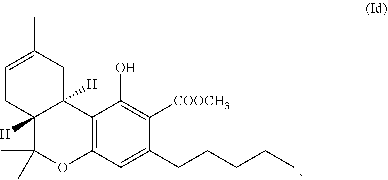 [SMILES: CCCCCc1cc2c(c(O)c1C(=O)OC)[C@@H]1CC(C)=CC[C@H]1C(C)(C)O2]

or a pharmaceutically acceptable salt thereof.

In some embodiments of formula (II), the compound is the cannabigerolic acid methyl ester (CBGA-Me) compound of formula (IIa):

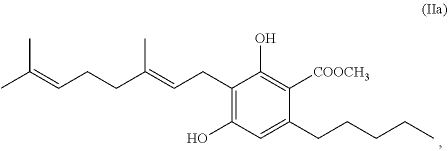 [SMILES: CCCCCc1cc(O)c(C/C=C(\C)CCC=C(C)C)c(O)c1C(=O)OC]

or a pharmaceutically acceptably salt thereof.

In some embodiments of formula (III), the compound is the cannabinolic acid methyl ester (CBNA-Me) compound of formula (IIIa):

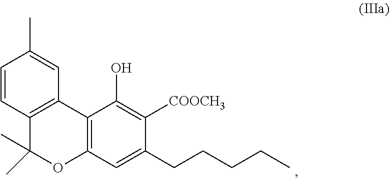 [SMILES: CCCCCc1cc2c(c(O)c1C(=O)OC)-c1cc(C)ccc1C(C)(C)O2]

or a pharmaceutically acceptable salt thereof.

Aspects of the present disclosure include compounds (e.g., as described herein), salts thereof (e.g., pharmaceutically acceptable salts), and/or solvate or hydrate forms thereof. It will be appreciated that all permutations of salts, solvates and hydrates are meant to be encompassed by the present disclosure. In some embodiments, the subject compounds are provided in the form of pharmaceutically acceptable salts.

### Pharmaceutical Compositions

The present disclosure provides pharmaceutical compositions including a cannabinoid acid ester compound (e.g., as described herein, such as a compound of any one of formulae (I)-(IX)), and a pharmaceutically acceptable excipient. The subject pharmaceutical compositions find use in a variety of therapeutic indications and other uses (e.g., as described herein).

In a first aspect, the present disclosure provides a pharmaceutical composition comprising:

a tetrahydrocannabinolic acid (THCA) ester compound of formula (I):

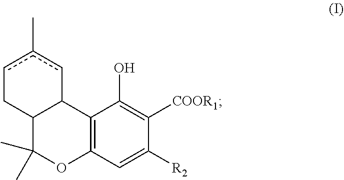

a cannabigerolic acid (CBGA) ester compound of formula (II):

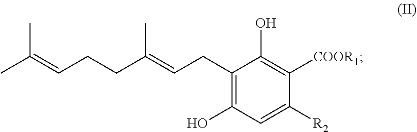

a cannabinolic acid (CBNA) ester compound of formula (III):

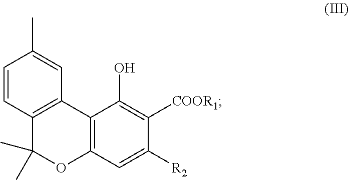

a combination thereof;

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof, and a pharmaceutically acceptable excipient.

In some embodiments of the composition, R₁ is C₁-C₆ alkyl or substituted C₁-C₆ alkyl. In some embodiments of the composition, R₁ is selected from methyl, ethyl, propyl, butyl, and pentyl. In some embodiments of the composition, R₁ is methyl.

In some embodiments of the composition, R₂ is C₁-C₁₀ alkyl, or substituted C₁-C₁₀ alkyl. In some embodiments of the composition, R₂ is C₂₋₆ alkyl. In some embodiments of the composition, R₂ is pentyl.

In some embodiments, the THCA ester compound of formula (I) is a THCA ester compound of formula (Ia):

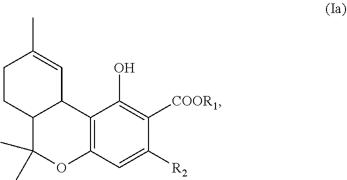

or a pharmaceutically acceptable salt thereof.

In some embodiments, the THCA ester compound of formula (I) is a THCA ester compound of formula (Ib):

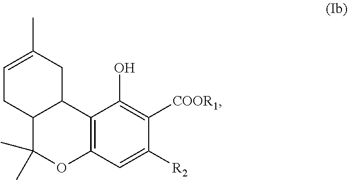

or a pharmaceutically acceptable salt thereof.

In some embodiments of the pharmaceutical composition, the THCA-Me compound is selected from an enantiomerically pure THCA-Me, a stereoisomeric mixture of THCA-Me, a diastereomeric mixture of THCA-Me, a salt thereof, a deuterated analog thereof, a fluorinated analog thereof, and a combination thereof.

In some embodiments, the THCA ester compound of formula (I) is the Δ⁹-tetrahydrocannabinolic acid methyl ester (Δ⁹-THCA-Me) compound of formula (Ic):

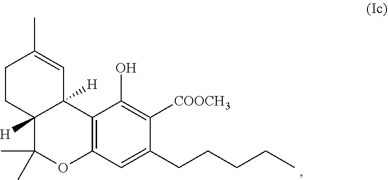 [SMILES: CCCCCc1cc2c(c(O)c1C(=O)OC)[C@@H]1C=C(C)CC[C@H]1C(C)(C)O2]

or a pharmaceutically acceptable salt thereof.

In some embodiments, the THCA ester compound of formula (I) is the Δ⁸-tetrahydrocannabinolic acid methyl ester (Δ⁸-THCA-Me) compound of formula (Id):

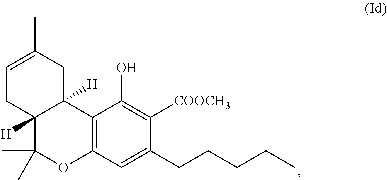 [SMILES: CCCCCc1cc2c(c(O)c1C(=O)OC)[C@@H]1CC(C)=CC[C@H]1C(C)(C)O2]

or a pharmaceutically acceptable salt thereof.

In some embodiments, the CBGA ester compound of formula (II) is the cannabigerolic acid methyl ester (CBGA-Me) compound of formula (IIa):

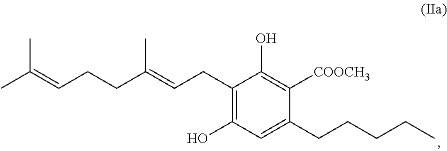 [SMILES: CCCCCc1cc(O)c(C/C=C(\C)CCC=C(C)C)c(O)c1C(=O)OC]

or a pharmaceutically acceptable salt thereof.

In some embodiments of the pharmaceutical composition, the CBGA-Me compound is selected from a diastereomeric mixture of CBGA-Me, a salt thereof, a deuterated analog thereof, a fluorinated analog thereof, and a combination thereof.

In some embodiments, the CBNA ester compound of formula (III) is the cannabinolic acid methyl ester (CBNA-Me) compound of formula (IIIa):

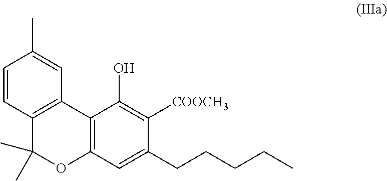 [SMILES: CCCCCc1cc2c(c(O)c1C(=O)OC)-c1cc(C)ccc1C(C)(C)O2]

or a pharmaceutically acceptable salt thereof.

According to some embodiments, the pharmaceutically acceptable excipient is an aqueous solution or carrier. In some embodiments the aqueous solution is a buffer having physiological or near-physiological pH, such as phosphate buffered saline (PBS). In some embodiments, the pharmaceutically acceptable excipient is selected from emulsifiers, buffering agents, pH adjusting agents, tonicity modifiers, preservatives, antioxidants, stabilizers, and a combination thereof. Exemplary excipients, additives and additional components of the subject pharmaceutical compositions are described in further detail below.

In some embodiments, the subject pharmaceutical composition can further include one or more additional active pharmaceutical ingredient (API). In another embodiment, the one or more additional API comprises one or more additional cannabinoid compound.

In some embodiments, the subject pharmaceutical composition can further include one or more additional cannabinoid compounds. The additional cannabinoid compound can be an isolated compound, or part of a complex mixture. The additional cannabinoid compound can be part of a crude component or composition ingredient, or a purified sample.

In some embodiments, the one or more additional cannabinoid compounds that are included into a pharmaceutical composition are independently comprised in one or more Cannabis plant extracts. The Cannabis plant extracts can be obtained from any convenient source. In some embodiments, the Cannabis plant extracts are produced from a plant strain selected from Cannabis sativa, Cannabis indica, Cannabis ruderalis, a hybrid strain, a strain with a high concentration of cannabidiol (CBD), a strain with a high concentration of tetrahydrocannabinol (THC), and a combination thereof.

In some embodiments, the one or more Cannabis plant extracts comprise a cannabinoid compound selected from cannabidiol (CBD), tetrahydrocannabinol (THC), cannabinol (CBN), cannabigerol (CBG), cannabichromene (CBC), acids thereof, esters of the acids thereof, and combinations thereof.

In some embodiments, the one or more additional cannabinoid compounds are independently selected from cannabidiol (CBD), cannbigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C₃ (THC-C₃), tetrahydrocannabinol-C₄ (THC-C₄), tetrahydrocannabinol-C₇ (THC-C₇), and esters thereof, stereoisomers thereof, deuterated analogs thereof, fluorinated analogs thereof, and combinations thereof.

In some embodiments, the one or more Cannabis plant extracts that are incorporated into a pharmaceutical composition include about 1% (w/w) or more of CBD, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more CBD. In certain embodiments, the one or more Cannabis plant extracts comprise about 30% (w/w) or less of CBD.

In some embodiments, the one or more Cannabis plant extracts comprise about 1% (w/w) or more of THC, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more THC. In certain embodiments, the one or more Cannabis plant extracts comprise about 30% (w/w) or less of THC.

In some embodiments, the one or more Cannabis plant extracts comprising the one or more additional cannabinoid compounds are produced by extraction from a Cannabis plant with a suitable solvent, or a combination of solvents. In some embodiments, the solvent is a pharmaceutically acceptable solvent, for example but not limited to, a non-toxic solvent that is suitable for administration to a mammal with no unacceptable adverse effects. The solvent may be an aqueous or non-aqueous solvent.

In some embodiments, the solvent for extraction is a polar solvent, a hydrocarbon solvent, an alcohol solvent, carbon dioxide, an oil, or a combination thereof.

In some embodiments, the solvent for extraction is a polar solvent.

In some embodiments, the solvent for extraction is polyethylene glycol or propylene glycol.

In some embodiments, the polar solvent for extraction comprises tetrahydrofuran (THF), dichloromethane (DCM), ethyl acetate (EtOAc), methanol, ethanol, propanol, isopropanol, butanol, tert-butanol, or dimethyl sulfoxide (DMSO).

In some embodiments, the solvent is an alcohol solvent. In some embodiments, the alcohol solvent is ethanol.

In some embodiments, the solvent for extraction is a hydrocarbon solvent comprising pentane, hexane, n-hexane, xylene, toluene, or benzene.

In some embodiments, the solvent for extraction is an oil comprising copaiba oil, vegetable oil, olive oil, sesame oil, coconut oil, avocado oil, peanut oil, canola oil, cottonseed oil, soybean oil, safflower oil, sunflower oil, castor oil, corn oil, palm oil, poppy seed oil, or walnut oil. In another embodiment, the pharmaceutical composition comprises copaiba oil.

In some embodiments, the pharmaceutical composition comprises 1% to 20% (w/w) a cannabinoid acid ester compound and 50% to 90% vegetable oil.

In some embodiments, the pharmaceutical composition comprises 1% to 10% (w/w) of the tetrahydrocannabinolic acid ester compound of formula (I).

In some embodiments, the THCA ester compound is the Δ⁹-THCA-Me ester compound of formula (Ic). In another embodiment, the pharmaceutical composition comprises 1% to 10% Δ⁹-THCA-Me, 20% to 60% alcohol, and 0% to 35% propylene glycol. In another embodiment, the pharmaceutical composition comprises 2% to 10% Δ⁹-THCA-Me, 30% to 60% ethanol, and a combination of propylene glycol and polyethylene glycol. In another embodiment, the pharmaceutical composition further comprises 5% to 20% polyethylene glycol.

In some embodiments, the THCA ester compound is the Δ⁸-THCA-Me ester compound of formula (Id). In another embodiment, the pharmaceutical composition comprises 1% to 10% Δ⁸-THCA-Me, 20% to 60% alcohol, and 0% to 35% propylene glycol. In another embodiment, the pharmaceutical composition comprises 2% to 10% Δ⁸-THCA-Me, 30% to 60% ethanol, and a combination of propylene glycol and polyethylene glycol. In another embodiment, the pharmaceutical composition further comprises 5% to 20% polyethylene glycol.

In some embodiments, the ratio of the THCA ester compound of formula (I) to additional cannabinoid compound in the composition is from 1.05:1 to 1,000:1.

In some embodiments, the pharmaceutical composition comprises 1% to 10% (w/w) of the cannabigerolic acid (CBGA) ester compound of formula (II).

In some embodiments, the CBGA ester compound is the CBGA-Me ester compound of formula (IIa). In another embodiment, the pharmaceutical composition comprises 1% to 10% CBGA-Me, 20% to 60% alcohol, and 0% to 35% propylene glycol. In another embodiment, the pharmaceutical composition comprises 2% to 10% CBGA-Me, 30% to 60% ethanol, and a combination of propylene glycol and polyethylene glycol. In another embodiment, the pharmaceutical composition further comprises 5% to 20% polyethylene glycol.

In another embodiment, the ratio of the CBGA ester compound of formula (II) to additional cannabinoid compounds in the composition is from 1.05:1 to 1,000:1.

In some embodiments, the pharmaceutical composition comprises 1% to 10% (w/w) of the cannabinolic acid (CBNA) ester compound of formula (III).

In some embodiments, the CBNA ester compound is the CBNA-Me ester compound of formula (IIIa). In another embodiment, the pharmaceutical composition comprises 1% to 10% CBNA-Me, 20% to 60% alcohol, and 0% to 35% propylene glycol. In another embodiment, the pharmaceutical composition comprises 2% to 10% CBNA-Me, 30% to 60% ethanol, and a combination of propylene glycol and polyethylene glycol. In another embodiment, the pharmaceutical composition further comprises 5% to 20% polyethylene glycol.

In some embodiments, the ratio of the CBNA ester compound of formula (III) to additional cannabinoid compounds in the composition is from 1.05:1 to 1,000:1.

In some embodiments, the pharmaceutical composition comprises a combination of cannabinoids, a pharmaceutical carrier, a co-solvent, a penetration enhancer, and an emulsifier. In another embodiment, the pharmaceutical composition comprises about 5% to 80% of the pharmaceutical carrier, about 50% of the co-solvent, about 1% to 5% of the penetration enhancer, about 0.1% to 20% of the emulsifier, and about 0.001% to 10% of the combination of cannabinoids.

In some embodiments, the pharmaceutical composition comprises about 5% to 60% of anhydrous lanolin, about 5% to 80% of olive oil, about 0.2% to 20% polysorbate, about 0.001% to 10% of the combination of cannabinoids, and jojoba oil.

In some embodiments, the pharmaceutical composition comprises about 5% to 60% of anhydrous lanolin, about 5% to 80% of white petrolatum, about 5% to 80% of white olive oil, about 0.2% to 20% polysorbate 80, and about 0.001% to 10% of the combination of cannabinoids.

In some embodiments, the pharmaceutical composition comprises about 0% to 50% of ethanol, about 5% to 30% of glycerol, about 10% to 90% of propylene glycol, and about 0.2% to 25% of cyclosporine A powder.

In some embodiments, the ratio of the cannabinoid acid ester compound of formula (I) to additional cannabinoid compounds in the pharmaceutical composition is from 1.05:1 to 1,000:1. In another embodiment, the ratio of the cannabinoid acid ester compound of formula (I) to additional cannabinoid compounds in the pharmaceutical composition is from 1.05:1 to 5:1. In another embodiment, the ratio of the cannabinoid acid ester compound of formula (I) to additional cannabinoid compounds in the pharmaceutical composition is from 5:1 to 50:1. In another embodiment, the ratio of the cannabinoid acid ester compound of formula (I) to additional cannabinoid compounds in the pharmaceutical composition is from 50:1 to 500:1. In another embodiment, the ratio of the cannabinoid acid ester compound of formula (I) to additional cannabinoid compounds in the pharmaceutical composition is from 500:1 to 1,000:1.

In some embodiments, the ratio of the cannabinoid acid ester compound of formula (II) to additional cannabinoid compounds in the pharmaceutical composition is from 1.05:1 to 1,000:1. In another embodiment, the ratio of the cannabinoid acid ester compound of formula (II) to additional cannabinoid compounds in the pharmaceutical composition is from 1.05:1 to 5:1. In another embodiment, the ratio of the cannabinoid acid ester compound of formula (II) to additional cannabinoid compounds in the pharmaceutical composition is from 5:1 to 50:1. In another embodiment, the ratio of the cannabinoid acid ester compound of formula (II) to additional cannabinoid compounds in the pharmaceutical composition is from 50:1 to 500:1. In another embodiment, the ratio of the cannabinoid acid ester compound of formula (II) to additional cannabinoid compounds in the pharmaceutical composition is from 500:1 to 1,000:1.

In some embodiments, the ratio of the cannabinoid acid ester compound of formula (III) to additional cannabinoid compounds in the pharmaceutical composition is from 1.05:1 to 1,000:1. In another embodiment, the ratio of the cannabinoid acid ester compound of formula (III) to additional cannabinoid compounds in the pharmaceutical composition is from 1.05:1 to 5:1. In another embodiment, the ratio of the cannabinoid acid ester compound of formula (III) to additional cannabinoid compounds in the pharmaceutical composition is from 5:1 to 50:1. In another embodiment, the ratio of the cannabinoid acid ester compound of formula (III) to additional cannabinoid compounds in the pharmaceutical composition is from 50:1 to 500:1. In another embodiment, the ratio of the cannabinoid acid ester compound of formula (III) to additional cannabinoid compounds in the pharmaceutical composition is from 500:1 to 1,000:1.

In some embodiments, the pharmaceutical composition further comprises a lipid. The lipid can be triglyceride, fat, oil, fatty acid, or a mixture thereof.

In another embodiment, the lipid is a phospholipid, preferably a naturally occurring phospholipid or a synthetic phospholipid. In another embodiment, the lipid is a naturally occurring phospholipid selected from soy lecithin, egg lecithin, hydrogenated soy lecithin, hydrogenated egg lecithin, and combinations thereof. In another embodiment, the lipid is a synthetic phospholipid selected from phosphocholines, phosphoethanolamines, phosphatidic acids, phosphoglycerols, phosphoserines, mixed chain phospholipids, lysophospholipids, pegylated phospholipids, and combinations thereof.

In some embodiments, the phospholipid may form micelles, emulsions, or liposomes. In some embodiments, the cannabinoid acid ester compounds as described herein are provided in microencapsulation particles. Encapsulation may result in cannabinoids and other materials present in cannabinoid materials in liposomal capsule particles or other type of particles.

In some embodiments, microencapsulation or nanoencapsulation may increase cannabinoid bioavailability, thereby increasing cannabinoid efficacy after absorption through mucosal membrane. Microencapsulation or nanoencapsulation may result in particles of 20-40 nm in size. Microencapsulation or nanoencapsulation promotes dissolution of cannabinoid particles in an aqueous environment.

In some embodiments, the pharmaceutical composition comprises at least one micelle-forming compound selected from a polyoxyethylene ether, ester, or alcohol; an alkali metal alkyl sulfate, a bile acid, lecithin, hyaluronic acid, pharmaceutically acceptable salts of hyaluronic acid, octylphenoxypolyethoxyethanol, glycolic acid, lactic acid, chamomile extract, cucumber extract, oleic acid, linolenic acid, borage oil, evening of primrose oil, trihydroxy oxo-cholanylglycine, glycerin, poly glycerin, lysine, polylysine, triolein, salts thereof, and mixtures thereof. According to certain embodiments, the bile acids or bile acid salts are selected from the group consisting of chenodesoxycholic acid (CDCA), desoxycholic acid (DCA), lithocholic acid (LCA), taurodesoxycholic acid (TDCA), hyodeoxycholic acid (HDCA), taurocholic acid (TCA), glycocholic acid (GCA), and combinations thereof.

In some embodiments, the pharmaceutical composition further comprises triglyceride, fat, oil, fatty acid, or a mixture thereof. In some embodiments of the pharmaceutical composition, the composition further comprises cyclodextrin, electrolyte, vitamin, mineral, flavoring agent, or a combination thereof.

In some embodiments, the pharmaceutical composition further comprises cyclodextrin. In another embodiment, the cyclodextrin is selected from hydroxypropyl β-cyclodextrin, sulfobutylether β-cyclodextrin, and methyl β-cyclodextrin (MPCD).

In some embodiments, the pharmaceutically acceptable excipient is aqueous. In another embodiment, the pharmaceutical acceptable excipient is selected from an emulsifier, a buffering agent, a pH adjusting agent, a preservative, an antioxidant, a stabilizer, an electrolyte, a vitamin, a mineral, a flavoring agent, a solubilizing agent, a tonicity enhancing agent, a colorant, and a combination thereof.

In some embodiments, the antioxidant is glycine, α-tocopherol or ascorbate, butylated hydroxyanisole (BHA), butylated hydroxytoluene (BHT), or a mixture thereof.

In some embodiments, the vitamin is selected from is vitamin A, vitamin C, vitamin D (e.g. vitamin D1, D2, D3, D4, and/or D5), vitamin E, vitamin B1 (thiamine), vitamin B2 (e.g. riboflavin), vitamin B3 (e.g. niacin or niacinamide), vitamin B5 (pantothenic acid), vitamin D6 (pyridoxine), vitamin D7 (biotin), vitamin B9 (e.g. folate or folic acid), vitamin B12 (cobalamin), vitamin K (e.g. K1, K2, K3, K4, and K5), and choline.

In some embodiments, the emulsifier is selected from polyvinyl alcohol (PVA), polysorbate, polyethylene glycols, polyoxyethylene-polyoxypropylene block copolymers, polyglycerin fatty acid esters, sorbitan fatty acid ester, polyoxyethylen sorbitan fatty acid ester, polyethylene glycol fatty acid ester, polyoxyethylene alkyl ether, polyoxyethylene castor oil, hardened castor oil, hydrogenated castor oil, sodium gluconate, acrylates, C₁₀₋₃₀ alkyl acrylate crosspolymer, sodium carboxymethyl betaglucan, polyglyceryl-3 methylglucose distearate, cetearyl alcohol, cetyl alcohol, stearic acid, behenyl alcohol, butylene glycol, propylene glycol, xanthan gum, potassium cetyl phosphate, polyglyceryl-6 distearate jojoba esters, polyglyceryl-3-beeswax, PEG-800, laureth-7, C₁₃₋₁₄ isoparaffin, polyisobutene, PEG-200 hydrogenated glyceryl palmate, cellulose gum, PEG-7 glyceryl cocoate, aluminum starch octenylsuccinate, and a combination thereof.

Solubilizing agent and emulsifiers can be used to improve the bioavailability of cannabinoid acid ester compounds as described herein. Bioavailability refers to the extent and rate at which the active moiety (drug or metabolite) enters systemic circulation, thereby accessing the site of action. Bioavailability for a given formulation provides an estimate of the relative fraction of the orally administered dose that is absorbed into the systemic circulation. Low bioavailability is most common with oral dosage forms of poorly water-soluble, slowly absorbed drugs. Insufficient time for absorption in the gastrointestinal tract is a common cause of low bioavailability. If the drug does not dissolve readily or cannot penetrate the epithelial membrane (e.g., if it is highly ionized and polar), time at the absorption site may be insufficient. Orally administered drugs must pass through the intestinal wall and then the portal circulation to the liver, both of which are common sites of first-pass metabolism (metabolism that occurs before a drug reaches systemic circulation).

In some embodiments, the bioavailability enhancing agent is an edible oil or fat, a protective colloid, or both a protective colloid and an edible oil or fat. In another embodiment, the bioavailability enhancing agent is also a lipophilic active agent taste masking agent. In another embodiment, the bioavailability of the lipophilic active agent in a subject is at last about 2 times, 5 times, or 10 times greater than the bioavailability of the lipophilic active agent in the subject in the absence of the bioavailability enhancing agent.

In some embodiments, the tonicity enhancing agent comprises ionic and non-ionic agents, such as alkali metal or alkaline earth metal halides, urea, glycerol, sorbitol, mannitol, propylene glycol, and dextrose.

In some embodiments, the preservative comprises chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, or polyhexamethylene biguanide.

In some embodiments, the flavoring agent comprises sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

In some embodiments, the colorant comprises alumina (dried aluminum hydroxide), annatto extract, calcium carbonate, canthaxanthin, caramel, β-carotene, cochineal extract, carmine, potassium sodium copper chlorophyllin (chlorophyllin copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

In some embodiments, the pH-adjusting agent is an organic or mineral acid.

In some embodiment of the pharmaceutical composition, the composition is formulated for administration orally, topically, systemically, intravenously, subcutaneously, nasally, rectally, intramuscularly, intraperitoneally, transdermally, intra-arterially, intranasally, vaginally, by vaporization, or by inhalation.

In some embodiment, the pharmaceutical composition is in a form selected from liquid, gel, cream, ointment, lotion, paste, tablet, pill, capsule, pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, and suppository.

In some embodiment, the pharmaceutical composition is formulated for oral administration. In some embodiments, the composition is formulated as a tablet or capsule. In some embodiments, the orally administered formulation is optionally coated or scored and may be formulated so as to provide sustained, delayed, or controlled release of the active ingredient therein.

In some embodiments, the formulation suitable for oral administration comprises a mixture of sodium carboxymethylcellulose and hydroxypropyl-cellulose or methyl cellulose as the film-forming agents. The ratio of sodium carboxymethylcellulose to hydroxypropyl cellulose (or methylcellulose) used to make the formulation is chosen to yield the desired dissolution time and mouth feel for the film and to further impart acceptable product handling characteristics. The formulation may include from about 5 wt % to 75 wt % particularly from about 15 to 50 wt %, based on the weight of the formulation of sodium carboxymethylcellulose and hydroxypropyl-cellulose (or methylcellulose). Pectin may also be combined with carboxymethylcellulose and hydroxypropyl-cellulose or methyl cellulose in an amount ranging from about 4 to 25 wt %.

In some embodiment, the pharmaceutical composition is formulated in the form of a liquid at a temperature in the range of 4° C. to 37° C.

In some embodiments, the pharmaceutical composition is formulated in the form of a gel at physiological temperature. In another embodiment, the pharmaceutical composition is a gel, wherein the cannabinoid component or salt thereof is entrapped in a gel matrix. In another embodiment, the gel compositions may comprise an oil-in-water (o/w) emulsion.

In some embodiments, the pharmaceutical composition is formulated for slow release of cannabinoid acid ester. In another embodiment, the pharmaceutical composition further comprises a release retarding agent or a mixture of release retarding agents. In another embodiment, the pharmaceutical composition is at least partly coated by an enteric-coating agent.

In some embodiments of the pharmaceutical composition, the composition further comprises emollient-based cream, keratolytic agent, coal tar ointment, steroid, vitamin D analog, anthralin, retinoid tazarotene, or a combination thereof.

In another embodiment, the keratolytic agent is formulated with urea or salicylic acid.

In some embodiment of the pharmaceutical composition, the composition further comprises antihistamine, anesthesia agent, terpene, or a combination thereof.

In some embodiments, the pharmaceutical composition further a viscosity agent.

In some embodiments, the viscosity agent is a polysaccharide, a polysaccharide salt, or a combination thereof. In another embodiment, the polysaccharide is selected from hyaluronic acid (HA), chitosan, cellulose derivative, chondroitin sulfate, keratan, heparin, xanthans, galactomann, alginates, and a combination thereof.

In some embodiments, the viscosity agent is present in the composition at a concentration in the range of 1 mg/ml to 100 mg/ml. In another embodiment, the viscosity agent is present in the composition at a concentration in the range of 10 mg/ml to 25 mg/ml.

In some embodiments, the viscosity of the pharmaceutical composition is up to 2,000 centipoises at 20° C. In another embodiment, the viscosity of the pharmaceutical composition is up to 2,000 centipoises at 20° C. In another embodiment, the viscosity of the pharmaceutical composition is up to 1,800 centipoises at 20° C. In another embodiment, the viscosity of the pharmaceutical composition is up to 1,600 centipoises at 20° C. In another embodiment, the viscosity of the pharmaceutical composition is up to 1,500 centipoises at 20° C. In another embodiment, the viscosity of the pharmaceutical composition is up to 2,000 centipoises at 20° C. In another embodiment, the viscosity of the pharmaceutical composition is up to 2,000 centipoises at 20° C.

In some embodiments, the viscosity of the pharmaceutical composition is less than 5,000 centipoises between 4° C. and 12° C. In another embodiment, the viscosity of the pharmaceutical composition is less than 500 centipoises between 4° C. and 12° C.

In some embodiments, the viscosity of the pharmaceutical composition is more than 500,000 centipoises at 37° C. In another embodiment, the viscosity of the pharmaceutical composition is more than 1×10³ centipoises at 37° C. In another embodiment, the viscosity of the pharmaceutical composition is more than 3.5×10³ at 37° C.

In some embodiments, the pharmaceutical composition comprises at least one physiologically acceptable film forming agent. In some embodiments, the physiologically acceptable film forming agent is selected from pullulan, methyl cellulose, ethyl cellulose, sodium carboxymethyl cellulose, hydroxypropylmethyl cellulose, hydroxyethyl cellulose, hydroxypropyl cellulose, carboxymethyl cellulose, polyvinyl pyrrolidone, methacrylic acid polymers, methacrylic acid copolymers, acrylic acid polymers, acrylic acid copolymers, polyacrylamides, polyalkylene oxides, carrageenan, polyvinyl alcohol, sodium alginate, polyethylene glycol, polyacrylic acid, glycolide, polylactide, methylmethacrylate copolymer, carboxyvinyl polymer, amylose, high amylose starch, hydroxypropylated high amylose starch, alginic acid, pea starch, dextrin, pectin, chitin, chitosan, levan, elsinan and mixtures thereof. Secondary film forming agents may be added to the formulation to optimize wafer characteristics such as tensile strength, stability, flexibility and brittleness including agents such xanthan gum, tragacanth gum, guar gum, locust bean gum, acacia gum, arabic gum, collagen, gelatin, zein, gluten, soy protein isolate, whey protein isolate, casein and a mixture thereof.

In some embodiments, the composition further comprises platelet-rich plasma (PRP) or platelet-rich fibrin (PRF).

In some embodiments, the PRP is selected from leukocyte-rich PRP (L-PRP), leukocyte reduced PRP and a combination thereof.

In some embodiments, the PRF is selected from leukocyte platelet-rich fibrin, pure platelet-rich fibrin, and a combination thereof.

In some embodiments, the pharmaceutical composition comprises 10% (w/w) or less of the tetrahydrocannabinolic acid ester compound of formula (I). In another embodiment, the pharmaceutical composition comprises 7% (w/w) or less of the tetrahydrocannabinolic acid ester compound of formula (I). In another embodiment, the pharmaceutical composition comprises 5% (w/w) or less of the tetrahydrocannabinolic acid ester compound of formula (I). In another embodiment, the pharmaceutical composition comprises 1% (w/w) or less of the tetrahydrocannabinolic acid ester compound of formula (I). In another embodiment, the pharmaceutical composition comprises 0.5% (w/w/) or less of the tetrahydrocannabinolic acid ester compound of formula (I). In another embodiment, the pharmaceutical composition comprises 0.1% (w/w) or less of tetrahydrocannabinolic acid ester compound of formula (I). In another embodiment, the pharmaceutical composition comprises 0.01% (w/w) or less of tetrahydrocannabinolic acid ester compound of formula (I). In another embodiment, the pharmaceutical composition comprises 0.001% (w/w) or less of tetrahydrocannabinolic acid ester compound of formula (I).

In some embodiments, the pharmaceutical composition comprises 10% (w/w) or less of the cannabigerolic acid ester compound of formula (II). In another embodiment, the pharmaceutical composition comprises 7% (w/w) or less of the cannabigerolic acid ester compound of formula (II). In another embodiment, the pharmaceutical composition comprises 5% (w/w) or less of the cannabigerolic acid ester compound of formula (II). In another embodiment, the pharmaceutical composition comprises 1% (w/w) or less of the cannabigerolic acid ester compound of formula (II). In another embodiment, the pharmaceutical composition comprises 0.5% (w/w/) or less of the cannabigerolic acid ester compound of formula (II). In another embodiment, the pharmaceutical composition comprises 0.1% (w/w) or less of cannabigerolic acid ester compound of formula (II). In another embodiment, the pharmaceutical composition comprises 0.01% (w/w) or less of cannabigerolic acid ester compound of formula (II). In another embodiment, the pharmaceutical composition comprises 0.001% (w/w) or less of cannabigerolic acid ester compound of formula (II).

In some embodiments, the pharmaceutical composition comprises 10% (w/w) or less of the cannabinolic acid ester compound of formula (III). In another embodiment, the pharmaceutical composition comprises 7% (w/w) or less of the cannabinolic acid ester compound of formula (III). In another embodiment, the pharmaceutical composition comprises 5% (w/w) or less of the cannabinolic acid ester compound of formula (III). In another embodiment, the pharmaceutical composition comprises 1% (w/w) or less of the cannabinolic acid ester compound of formula (III). In another embodiment, the pharmaceutical composition comprises 0.5% (w/w/) or less of the cannabinolic acid ester compound of formula (I III In another embodiment, the pharmaceutical composition comprises 0.1% (w/w) or less of cannabinolic acid ester compound of formula (III). In another embodiment, the pharmaceutical composition comprises 0.01% (w/w) or less of cannabinolic acid ester compound of formula (III). In another embodiment, the pharmaceutical composition comprises 0.001% (w/w) or less of cannabinolic acid ester compound of formula (III).

### Methods of Treatment

Aspects of the present disclosure include methods of treating, alleviating, or attenuating a target disease or disorder (e.g., as described herein) that include administration of a subject cannabinoid acid ester compound (e.g., as described herein) or subject pharmaceutical composition (e.g., as described herein) to a subject in need thereof.

In some embodiments, the disease, disorder or symptom is associated with phytocannabinoid receptor CB₁. In another embodiment, the disease, disorder or symptom is associated with phytocannabinoid CB₂. In another embodiment, the disease, disorder, or symptom is associated with phytocannabinoid receptor GPR55. In another embodiment, the disease, disorder, or symptoms is associated with phytocannabinoid receptor 5HT3A. In yet another embodiment, the disease, disorder, or symptom is associated with phytocannabinoid receptor PPARγ.

Administration of the subject cannabinoid acid ester compound (e.g., as described herein) or subject pharmaceutical composition can be performed via any convenient methods depending on the target indication and subject. In some embodiments, the pharmaceutical composition is administered orally. In another embodiment, the composition is administered topically. In another embodiment, the pharmaceutical composition is administered systemically. In another embodiment, the pharmaceutical composition is administered intravenously. In another embodiment, the pharmaceutical composition is administered subcutaneously. In another embodiment, the pharmaceutical composition is administered inhalation. In another embodiment, the pharmaceutical composition is administered vaporization. In another embodiment, the pharmaceutical composition is administered nasally. In another embodiment, the pharmaceutical composition is administered rectally. In another embodiment, the pharmaceutical composition is administered topically. In another embodiment, the pharmaceutical composition is administered intramuscularly. In another embodiment, the pharmaceutical composition is administered intraperitoneally. In another embodiment, the pharmaceutical composition is administered transdermally. In another embodiment, the pharmaceutical composition is administered intra-arterially. In another embodiment, the pharmaceutical composition is administered intranasally. In another embodiment, the pharmaceutical composition is administered vaginally. In some embodiments, the pharmaceutical composition is embedded in an article.

It is understood that the amount of compound or composition administered will be determined by a physician, according to various parameters including the chosen route of administration, the age, weight, and the severity of the subject's symptoms for the target disease.

The dosage and regimen will vary depending on the target indication and subject being treated. In some embodiments, the pharmaceutical composition is administered once a day, twice a day, three times a day, or four times a day. In some embodiments, the pharmaceutical composition is administered once a week, once in two weeks, once in three weeks, or once in a month. In some embodiments, the pharmaceutical composition is administered once in two months, once in three months, once in four months, once in five months, or once in six months.

In some embodiments, the pharmaceutical composition is administered at least 30 minutes before ingestion of food. In another embodiment, the pharmaceutical composition is administered at least one hour, two hours, or three hours before ingestion of food.

In some embodiments, the pharmaceutical composition is administered for a period of greater than a week. In some embodiments, the pharmaceutical composition is administered for a period of greater than four weeks. In some embodiments, the pharmaceutical composition is administered for a period greater than two months. In another embodiment, the pharmaceutical composition is administered for a period greater than 3, 4, 5, or 6 months.

In some embodiments, the effective dose of tetrahydrocannabinolic acid ester ranges from 0.1 to 500 mg/kg/day of body weight, from 1 to 250 mg/kg/day of body weight, from 2 to 100 mg/kg/day of body weight, or from 5 to 50 mg/kg/day, and may be in single dose or divided throughout the day.

In some embodiments, the effective dose of cannabigerolic acid ester ranges from 0.1 to 500 mg/kg/day of body weight, from 1 to 250 mg/kg/day of body weight, from 2 to 100 mg/kg/day of body weight, or from 5 to 50 mg/kg/day, and may be in single dose or divided throughout the day.

In some embodiments, the effective dose of cannabinolic acid ester ranges from 0.1 to 500 mg/kg/day of body weight, from 1 to 250 mg/kg/day of body weight, from 2 to 100 mg/kg/day of body weight, or from 5 to 50 mg/kg/day, and may be in single dose or divided throughout the day.

In some embodiments, the pharmaceutical composition is administered in a unit dosage form of approximately 0.02 g/kg/day to approximately 0.5 g/kg/day. In another embodiment, the unit dosage form is administered with food at any time of the day, without food at any time of the day, or with food after an overnight fast (e.g. with breakfast).

In some embodiments, the pharmaceutical composition is administered in combination with one or more additional therapeutic agents.

In some embodiments, the pharmaceutical composition may include ingredients in additional to the excipients described herein.

In some embodiments, the pharmaceutical composition is in a dosage unit. In some embodiments, the dosage unit comprises the active pharmaceutical ingredient as described herein and one or more additional therapeutic agents. In some embodiments, the one or more therapeutic agents of the dosage unit optionally exist in an extended or controlled release formulation. In some embodiments, the cannabinoid compounds as described herein may exist in a controlled release formulation or extended release formulation in the same dosage unit with another agent that may or may not be in either a controlled release or extended release formulation. In some embodiments, the unit dosage comprises one or more therapeutic agents as described herein formulated for immediate release and one or more other therapeutic agents as described herein formulated for controlled or extended release.

In some embodiments, the pharmaceutical composition comprises a unit dosage form of at least about 20 mg of cannabinoid acid ester or a mixture of cannabinoids comprising cannabinoid acid ester. In another embodiment, the unit dosage form comprises about 20 mg to about 2,000 mg of cannabinoid acid ester or a mixture of cannabinoids comprising cannabinoid acid ester.

In some embodiments, the pharmaceutical composition comprises a unit dosage form of at least about 20 mg of tetrahydrocannabinolic acid ester or a mixture of cannabinoids comprising tetrahydrocannabinolic acid ester. In another embodiment, the unit dosage form comprises about 20 mg to about 2,000 mg of tetrahydrocannabinolic acid ester or a mixture of cannabinoids comprising tetrahydrocannabinolic acid ester. In certain embodiments, the unit dosage form comprises about 50 mg, 100 mg, about 200 mg, about 300 mg, about 400 mg, about 500 mg, about 600, about 700 mg, about 800 mg, about 900 mg, or about 1000 mg of tetrahydrocannabinolic acid ester or a mixture of cannabinoids comprising tetrahydrocannabinolic acid ester.

In some embodiments, the pharmaceutical composition comprises a unit dosage form of at least about 20 mg of cannabigerolic acid ester or a mixture of cannabinoids comprising cannabigerolic acid ester. In another embodiment, the unit dosage form comprises about 20 mg to about 2,000 mg of cannabigerolic acid ester or a mixture of cannabinoids comprising cannabigerolic acid ester. In certain embodiments, the unit dosage form comprises about 50 mg, 100 mg, about 200 mg, about 300 mg, about 400 mg, about 500 mg, about 600, about 700 mg, about 800 mg, about 900 mg, or about 1000 mg of cannabigerolic acid ester or a mixture of cannabinoids comprising cannabigerolic acid ester.

In some embodiments, the pharmaceutical composition comprises a unit dosage form of at least about 20 mg of cannabinolic acid ester or a mixture of cannabinoids comprising cannabinolic acid ester. In another embodiment, the unit dosage form comprises about 20 mg to about 2,000 mg of cannabinolic acid ester or a mixture of cannabinoids comprising cannabinolic acid ester. In certain embodiments, the unit dosage form comprises about 50 mg, 100 mg, about 200 mg, about 300 mg, about 400 mg, about 500 mg, about 600, about 700 mg, about 800 mg, about 900 mg, or about 1000 mg of cannabinolic acid ester or a mixture of cannabinoids comprising cannabinolic acid ester.

Further aspects of the methods of using the subject cannabinoid compounds and pharmaceutical compositions comprising the same are discussed below.

### Pain

Pain disorder is acute and/or chronic pain experienced by a patient in one or more areas, and is thought to be caused by various factors, including but are not limited to psychological stress. Pain disorder can disable the patient from proper functioning. Duration of pain may be as short as a few days or as long as many years. Pain may be experience in a subject beginning at any age and regardless of gender. Pain disorder can occur during an illness that causes pain. In some cases, the pain disorder is caused by multiple sclerosis, Fibromyalgia, cancer, and peripheral neuropathy.

Aspects of the present disclosure include methods of treating a pain disorder or symptom using the subject compounds and pharmaceutical compositions. The method includes administering a therapeutically effective amount of the pharmaceutical composition as described herein to a subject having a pain disorder or symptom.

In some embodiments, the composition disclosed herein is for use in the treatment of pain. In another embodiment, the composition disclosed herein is for use in the attenuation of pain. In another embodiment, the composition disclosed herein is for use in the alleviation of pain.

### Impaired Neurological Function

Neurological disorders feature nervous system (neurological) symptoms and are related to how the brain functions. Signs and symptoms vary depending on the type of functional neurological disorder and may include specific patterns. Typically, these disorders affect a patient's movement or senses, such as ability to walk, swallow, see or hear. Symptoms can vary in severity and may come and go or be persistent, and include but are not limited to, weakness or paralysis, abnormal movement such as tremors or difficult in walking, loss of balance, difficulty swallowing, seizures or episodes of shaking and apparent loss of consciousness such as nonepileptic seizures, episodes of unresponsiveness, numbness or loss of touch sensations, speech, vision and hearing problems.

The causes of functional neurological disorders are unknown, and the conditions may be triggered by a neurological disorder or by a reaction to stress or psychological or physical trauma. Functional neurological disorders are related to how the brain function.

Aspects of the present disclosure include methods of treating neurological disorder or disease using the subject compounds and pharmaceutical compositions. The method includes administering a therapeutically effective amount of the pharmaceutical composition as described herein to a subject having a pain disorder or symptom.

In some embodiments, the composition disclosed herein is for use in the treatment of impaired neurological function selected from the group consisting of stroke, trauma, Parkinson's Disease, vascular dementia, senile dementia, Alzheimer's disease, mild cognitive impairment, Huntington's Disease, Amyotrophic Lateral Sclerosis (ALS), epilepsy, multiple sclerosis, and psychiatric disorders.

In some embodiments, the impaired neurological function is epilepsy.

In some embodiments, administration of the pharmaceutical composition improves the impaired neurological function of the subject. In another embodiment, the pharmaceutical composition improves the subject's movement, such as ability to walk and swallow, senses, such as ability to see and hear, balance, seizure episodes, episodes of shaking and apparent loss of consciousness such as nonepileptic seizures, episodes of unresponsiveness, numbness or loss of touch sensations, speech, vision and hearing problems. In another embodiment, the pharmaceutical composition reduces the number of seizure episodes. In another embodiment, the pharmaceutical composition reduces the frequency of shaking and apparent loss of consciousness episodes. In another embodiment, the pharmaceutical composition reduces the frequency of nonepileptic seizures. In yet another embodiment, the pharmaceutical composition reduces the frequency of unresponsiveness episodes.

In some embodiments, the psychiatric disorder is selected from the group consisting of depression, anxiety, acute or chronic stress, schizophrenia, panic attacks, ADHA, bipolar disorder, obsessive compulsive disorder, and personality disorders.

In some embodiments, the administration of the pharmaceutical composition improves the psychiatric disorder and its symptoms thereof.

In some embodiments of the method, the pharmaceutical composition is administered once a day, twice a day, three times a day, or four times a day. In another embodiment, the pharmaceutical composition is administered once in two days in a week, once in three days in a week, once in four days in a week, or once in five days in a week.

In some embodiments, the pharmaceutical composition is administered topically in a therapeutically effective amount. In another embodiment, the therapeutically effective amount of the pharmaceutical composition is administered up to about four times a day. In another embodiment, the therapeutically effective amount of the pharmaceutical composition is administered twice a day. In another embodiment, the therapeutically effective amount of the pharmaceutical composition is administered twice daily at intervals of about 12 hours. In another embodiment, the therapeutically effective amount of the pharmaceutical composition is administered once a day.

In some embodiments of the method, the pharmaceutical composition is administered in combination with one or more additional therapeutic agents suitable for treating a neurological disease or disorder.

In some embodiments of the method, the pharmaceutical composition is administered in combination with one or more additional therapeutic agents suitable for treating a neurological disease or disorder.

### Autoimmune Diseases and Inflammation

Autoimmune diseases occur when the immune system mistakes its own tissues as foreign and mounts an inappropriate attach on the body. Overblown inflammation is a common thread in these chronic conditions. Examples of autoimmune disease include, but is not limited to, multiple sclerosis, type 1 diabetes, Crohn's disease, lupus, and rheumatoid arthritis. In autoimmune disease, overproduction of cytokines and chemokines lead to inflammation of body tissues. This condition worsens when chemokines summon more destructive immune system components, cells such as macrophages, neutrophils, and T-cells, to the site of inflammation, amplifying the inflammatory response.

Aspects of the present disclosure include methods of treating inflammation and autoimmune diseases using the subject compounds and pharmaceutical compositions. The method includes administering a therapeutically effective amount of the pharmaceutical composition as described herein to a subject having inflammation and/or autoimmune diseases.

In some embodiments, the autoimmune disease is selected from type 1 diabetes, rheumatoid arthritis (RA), psoriasis/psoriatic arthritis, multiple sclerosis, systemic lupus erythematosus (SLE), inflammatory bowel disease (IBD), Addison's disease, Graves' disease, Sjogren's syndrome, Hashimoto's thyroiditis, Myasthenia gravis, autoimmune vasculitis, pernicious anemia, and Celiac disease.

In some embodiments, the symptom of autoimmune disease is selected from fatigue, achy muscles, swelling and redness, low-grade fever, difficulty concentrating, numbness and tingling in the hands and feet, hair loss, and skin rashes.

In some embodiments, the present disclosure provides methods for treating inflammatory skin diseases or disorders. In certain embodiments, the inflammatory skin disease or disorder is selected from psoriasis, atopic dermatitis (AD), eczema, actinic keratosis, ichthyosis, pemphigus vulgaris, acne, Grover's disease (transient acantholytic dermatosis), keratoacanthoma, hidradenitis suppurativa, seborrheic keratosis, pityriasis lichenoid, alopecia areata, basal cell carcinoma, Bowen's disease, congenital erythropoietic porphyria, contact dermatitis, Darier's disease, dystrophic epidermolysis bullosa, pidermolysis bullosa simplex, erythropoietic protoporphyria, fungal infections of nails, herpes simplex, hidradenitis suppurativa, ichthyosis, impetigo, keloids, keratosis pilaris, lichen planus, lichen sclerosus, pemphigus vulgaris, plantar warts (verrucas), pityriasis lichenoides, polymorphic light eruption, pyoderma gangrenosum, rosacea, shingles, squamous cell carcinoma, Sweet's syndrome, and vitiligo. In another embodiment, the inflammatory skin disease is caused by microbial infection-induced dermatitis, solar dermatitis, atopic dermatitis, or allergic contact dermatitis.

In some embodiments of the method, the skin disease or disorder is epidermal hyperproliferation or dermal inflammation.

In some embodiments, the method reduces or at least alleviates the symptoms associated with a skin disease or disorder. In another embodiment, the method reduces pain associated with skin disease or disorder.

In some embodiments, the pharmaceutical composition is formulated for topical administration. In another embodiment, the pharmaceutical composition is formulated in a form selected from a cream, an ointment, a lotion, a paste, and a gel. In some embodiments, the pharmaceutical composition is formulated as a cream.

In some embodiments of the method, the pharmaceutical composition is administered once a day, twice a day, three times a day, or four times a day. In another embodiment, the pharmaceutical composition is administered once in two days in a week, once in three days in a week, once in four days in a week, or once in five days in a week.

In some embodiments, the pharmaceutical composition is administered topically in a therapeutically effective amount. In another embodiment, the therapeutically effective amount of the pharmaceutical composition is administered up to about four times a day. In another embodiment, the therapeutically effective amount of the pharmaceutical composition is administered twice a day. In another embodiment, the therapeutically effective amount of the pharmaceutical composition is administered twice daily at intervals of about 12 hours. In another embodiment, the therapeutically effective amount of the pharmaceutical composition is administered once a day.

In some embodiments of the method, the pharmaceutical composition is administered in combination with one or more additional therapeutic agents suitable for treating or preventing a skin disease or disorder.

### Nausea, Vomiting or Low Appetite

Nausea or a loss of appetite may be experienced by a subject or patient because of a medical condition or the treatment they are receiving. Nausea is often a feeling in the mouth, back of the throat, and stomach that many people refer to as feeling sick. Nausea can sometimes lead to vomiting. Other symptoms that can accompany nausea are dizziness, difficulty swallowing, excess saliva production, and clammy skin. Loss of appetite can refer to not feeling hungry, eating much less than usual, or not eating at all. A person may have no interested in food or not want to eat. Although it is often a short-term, prolonged appetite loss can result in weight loss, tiredness, and dehydration. In some embodiments, causes of nausea, vomiting and/or loss in appetite include, but is not limited to, colds, flu, respiratory infections, bacteria or viral infections, constipation, upset stomach, digestive issues, acid reflux, food poisoning, food intolerances, stomach bug or gastroenteritis, pregnancy, hormonal imbalances, stress, medication side effects, alcohol consumptions, drug use, asthma, diabetes, chronic liver or kidney disease, high calcium levels in the blood, HIV, AIDS, underactive thyroid or hypothyroidism, overactive thyroid or hyperthyroidism, COPD, heart failure, stomach cancer, and colon cancer.

Certain medical treatments can also induce nausea, vomiting, and/or low appetite in the subjects receiving such medical treatments. In some embodiments, the medical treatment that may lead to nausea, vomiting, and/or low appetite in a subject include, but is not limited to, chemotherapy.

In some embodiments, the methods described herein treats, alleviates and/or attenuates nausea, vomiting, and/or loss of appetite and their symptoms thereof.

In some embodiments of the method, the pharmaceutical composition is administered once a day, twice a day, three times a day, or four times a day. In another embodiment, the pharmaceutical composition is administered once in two days in a week, once in three days in a week, once in four days in a week, or once in five days in a week.

In some embodiments of the method, the pharmaceutical composition is administered in combination with one or more additional therapeutic agents suitable for treating or preventing a skin disease or disorder.

### Glaucoma

Glaucoma is an eye condition that damage the optic nerve which is vital for good vision. This damage is often caused by an abnormally high pressure in the eye. Glaucoma is one of the leading causes for blindness for people over the age of 60, but it can also occur at any age regardless of race or gender. Many forms of glaucoma have no warning signs since the effect is gradual and the change in vision might undetected until the condition is at an advanced stage. Vision loss due to glaucoma is irreversible but can be slowed and prevent if diagnosed in its early stages.

Glaucoma is the result of damage to the optic nerve, which is related to increased pressure in the eye. Elevated eye pressure is due to a buildup of a fluid (aqueous humor) that flows throughout the inside of your eye. As the optic nerve gradually deteriorates, blind spots develop in the visual field. Some types of glaucoma include, but are not limited to, open-angle glaucoma, angle-closure glaucoma, normal-tension glaucoma, pigmentary glaucoma, and glaucoma in children.

Symptoms of glaucoma include, but are not limited to, open-angle glaucoma symptoms where patchy blind spots in peripheral or central vision in both eyes, or tunnel vision is observed, and acute angle-closure glaucoma symptoms such as severe headache, eye pain, nausea and vomiting, blurred vision, halos around lights, and eye redness.

In some embodiments, the methods described herein treats, alleviates and/or attenuates glaucoma and its symptoms thereof.

In some embodiments of the method, the pharmaceutical composition is administered once a day, twice a day, three times a day, or four times a day. In another embodiment, the pharmaceutical composition is administered once in two days in a week, once in three days in a week, once in four days in a week, or once in five days in a week.

In some embodiments of the method, the pharmaceutical composition is administered in combination with one or more additional therapeutic agents suitable for treating, alleviating, and/or attenuates glaucoma and symptoms thereof.

### Cosmetic Compositions

The present disclosure also provides cosmetic compositions including a cannabinoid acid ester compound (e.g., as described herein, such as a compound of any one of formulae (I)-(IX)), and a physiological acceptable excipient. The subject cosmetic compositions find use in a variety of therapeutic indications and other uses (e.g., as described herein).

Accordingly, the present disclosure provides a cosmetic composition comprising:

a tetrahydrocannabinolic acid (THCA) ester compound of formula (I);

a cannabigerolic acid (CBGA) ester compound of formula (II);

a cannabinolic acid (CBNA) ester compound of formula (III); or

a combination thereof;

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a physiologically acceptable salt thereof, and a physiologically acceptable excipient.

In some embodiments of the composition, R₁ is C₁-C₆ alkyl or substituted C₁-C₆ alkyl. In some embodiments of the composition, R₁ is selected from methyl, ethyl, propyl, butyl, and pentyl. In some embodiments of the composition, R₁ is methyl.

In some embodiments of the composition, R₂ is C₁-C₁₀ alkyl, or substituted C₁-C₁₀ alkyl. In some embodiments of the composition, R₂ is C₂₋₆ alkyl. In some embodiments of the composition, R₂ is pentyl.

In some embodiments, the THCA ester compound of formula (I) is a THCA ester compound of formula (Ia), or a physiologically acceptable salt thereof.

In some embodiments, the THCA ester compound of formula (I) is a THCA ester compound of formula (Ib), or a physiologically acceptable salt thereof.

In some embodiments of the cosmetic composition, the THCA-Me compound is selected from an enantiomerically pure THCA-Me, a stereoisomeric mixture of THCA-Me, a diastereomeric mixture of THCA-Me, a salt thereof, a deuterated analog thereof, a fluorinated analog thereof, and a combination thereof.

In some embodiments, the THCA ester compound of formula (I) is the Δ⁹-tetrahydrocannabinolic acid methyl ester (Δ⁹-THCA-Me) compound of formula (Ic), or a physiologically acceptable salt thereof.

In some embodiments, the THCA ester compound of formula (I) is the Δ⁸-tetrahydrocannabinolic acid methyl ester (Δ⁸-THCA-Me) compound of formula (Id), or a physiologically acceptable salt thereof.

In some embodiments, the CBGA ester compound of formula (II) is the cannabigerolic acid methyl ester (CBGA-Me) compound of formula (IIa), or a physiologically acceptable salt thereof.

In some embodiments of the cosmetic composition, the CBGA-Me compound is selected from a diastereomeric mixture of CBGA-Me, a salt thereof, a deuterated analog thereof, a fluorinated analog thereof, and a combination thereof.

In some embodiments, the CBNA ester compound of formula (III) is the cannabinolic acid methyl ester (CBNA-Me) compound of formula (IIIa), or a physiologically acceptable salt thereof.

According to some embodiments, the physiologically acceptable excipient is an aqueous solution or carrier. In some embodiments, the aqueous solution is a buffer having physiological or near-physiological pH, such as phosphate buffered saline (PBS). In some embodiments, the physiological acceptable excipient is selected from emulsifiers, buffering agents, pH adjusting agents, tonicity modifiers, preservatives, antioxidants, stabilizers, and a combination thereof. Exemplary excipients, additives and additional components of the subject physiological compositions are described in further detail below.

In some embodiments, the subject cosmetic composition can further include one or more additional cannabinoid compounds. The additional cannabinoid compound can be an isolated compound, or part of a complex mixture. The additional cannabinoid compound can be part of a crude component or composition ingredient, or a purified sample.

In some embodiments, the one or more additional cannabinoid compounds that are included into a cosmetic composition are independently comprised in one or more Cannabis plant extracts. The Cannabis plant extracts can be obtained from any convenient source. In some embodiments, the Cannabis plant extracts are produced from a plant strain selected from Cannabis sativa, Cannabis indica, Cannabis ruderalis, a hybrid strain, a strain with a high concentration of cannabidiol (CBD), a strain with a high concentration of tetrahydrocannabinol (THC), and a combination thereof.

In some embodiments, the one or more Cannabis plant extracts comprise a cannabinoid compound selected from cannabidiol (CBD), tetrahydrocannabinol (THC), cannabinol (CBN), cannabigerol (CBG), cannabichromene (CBC), acids thereof, esters of the acids thereof, and combinations thereof.

In some embodiments, the one or more additional cannabinoid compounds are independently selected from cannabidiol (CBD), cannbigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C₃ (THC-C₃), tetrahydrocannabinol-C₄ (THC-C₄), tetrahydrocannabinol-C₇ (THC-C₇), and esters thereof, stereoisomers thereof, deuterated analogs thereof, fluorinated analogs thereof, and combinations thereof.

In some embodiments, the one or more Cannabis plant extracts that are incorporated into a cosmetic composition include about 1% (w/w) or more of CBD, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more CBD. In certain embodiments, the one or more Cannabis plant extracts comprise about 30% (w/w) or less of CBD.

In some embodiments, the one or more Cannabis plant extracts comprise about 1% (w/w) or more of THC, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more THC. In certain embodiments, the one or more Cannabis plant extracts comprise about 30% (w/w) or less of THC.

In some embodiments, the one or more Cannabis plant extracts comprising the one or more additional cannabinoid compounds are produced by extraction from a Cannabis plant with a suitable solvent, or a combination of solvents. In some embodiments, the solvent is a physiologically acceptable solvent, for example but not limited to, a non-toxic solvent that is suitable for administration to a mammal with no unacceptable adverse effects. The solvent may be an aqueous or non-aqueous solvent.

In some embodiments, the solvent for extraction is a polar solvent, a hydrocarbon solvent, an alcohol solvent, carbon dioxide, an oil, or a combination thereof.

In some embodiments, the solvent for extraction is an oil comprising copaiba oil, vegetable oil, olive oil, sesame oil, coconut oil, avocado oil, peanut oil, canola oil, cottonseed oil, soybean oil, safflower oil, sunflower oil, castor oil, corn oil, palm oil, poppy seed oil, or walnut oil. In another embodiment, the cosmetic composition comprises copaiba oil.

In some embodiments, the cosmetic composition comprises 1% to 20% (w/w) a cannabinoid acid ester compound and 50% to 90% vegetable oil.

In some embodiments, the cosmetic composition comprises 1% to 10% (w/w) of the tetrahydrocannabinolic acid ester compound of formula (I).

In some embodiments, the THCA ester compound is the Δ⁹-THCA-Me ester compound of formula (Ic). In another embodiment, the cosmetic composition comprises 1% to 10% Δ⁹-THCA-Me, 20% to 60% alcohol, and 0% to 35% propylene glycol. In another embodiment, the cosmetic composition comprises 2% to 10% Δ⁹-THCA-Me, 30% to 60% ethanol, and a combination of propylene glycol and polyethylene glycol. In another embodiment, the cosmetic composition further comprises 5% to 20% polyethylene glycol.

In some embodiments, the THCA ester compound is the Δ⁸-THCA-Me ester compound of formula (Id). In another embodiment, the cosmetic composition comprises 1% to 10% Δ⁸-THCA-Me, 20% to 60% alcohol, and 0% to 35% propylene glycol. In another embodiment, the cosmetic composition comprises 2% to 10% Δ⁸-THCA-Me, 30% to 60% ethanol, and a combination of propylene glycol and polyethylene glycol. In another embodiment, the cosmetic composition further comprises 5% to 20% polyethylene glycol.

In some embodiments, the ratio of the THCA ester compound of formula (I) to additional cannabinoid compound in the composition is from 1.05:1 to 1,000:1.

In some embodiments, the cosmetic composition comprises 1% to 10% (w/w) of the cannabigerolic acid (CBGA) ester compound of formula (II).

In some embodiments, the CBGA ester compound is the CBGA-Me ester compound of formula (IIa). In another embodiment, the cosmetic composition comprises 1% to 10% CBGA-Me, 20% to 60% alcohol, and 0% to 35% propylene glycol. In another embodiment, the cosmetic composition comprises 2% to 10% CBGA-Me, 30% to 60% ethanol, and a combination of propylene glycol and polyethylene glycol. In another embodiment, the cosmetic composition further comprises 5% to 20% polyethylene glycol.

In another embodiment, the ratio of the CBGA ester compound of formula (II) to additional cannabinoid compounds in the composition is from 1.05:1 to 1,000:1.

In some embodiments, the cosmetic composition comprises 1% to 10% (w/w) of the cannabinolic acid (CBNA) ester compound of formula (III).

In some embodiments, the CBNA ester compound is the CBNA-Me ester compound of formula (IIIa). In another embodiment, the cosmetic composition comprises 1% to 10% CBNA-Me, 20% to 60% alcohol, and 0% to 35% propylene glycol. In another embodiment, the cosmetic composition comprises 2% to 10% CBNA-Me, 30% to 60% ethanol, and a combination of propylene glycol and polyethylene glycol. In another embodiment, the cosmetic composition further comprises 5% to 20% polyethylene glycol.

In some embodiments, the ratio of the CBNA ester compound of formula (III) to additional cannabinoid compounds in the composition is from 1.05:1 to 1,000:1.

In some embodiments, the cosmetic composition further comprises a lipid. The lipid can be triglyceride, fat, oil, fatty acid, or a mixture thereof.

In some embodiments, the cosmetic composition further comprises triglyceride, fat, oil, fatty acid, or a mixture thereof. In some embodiments of the pharmaceutical composition, the composition further comprises cyclodextrin, electrolyte, vitamin, mineral, flavoring agent, or a combination thereof.

In some embodiments, the cosmetic composition further comprises cyclodextrin. In another embodiment, the cyclodextrin is selected from hydroxypropyl β-cyclodextrin, sulfobutylether β-cyclodextrin, and methyl β-cyclodextrin (MPCD).

In some embodiments, the physiologically acceptable excipient is aqueous. In another embodiment, the physiologically acceptable excipient is selected from an emulsifier, a buffering agent, a pH adjusting agent, a preservative, an antioxidant, a stabilizer, an electrolyte, a vitamin, a mineral, a flavoring agent, a solubilizing agent, a tonicity enhancing agent, a colorant, and a combination thereof.

In some embodiments, the colorant comprises alumina (dried aluminum hydroxide), annatto extract, calcium carbonate, canthaxanthin, caramel, β-carotene, cochineal extract, carmine, potassium sodium copper chlorophyllin (chlorophyllin copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

In some embodiments, the pH-adjusting agent is an organic or mineral acid.

In some embodiment of the cosmetic composition, the composition is formulated for administration topically or transdermally.

In some embodiment, the cosmetic composition is in a form selected from liquid, gel, cream, ointment, lotion, paste, tablet, pill, capsule, pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, and suppository.

In some embodiment, the cosmetic composition is formulated at a temperature in the range of 4° C. to 37° C.

In some embodiments, the cosmetic composition is formulated in the form of a gel at physiological temperature. In another embodiment, the cosmetic composition is a gel, wherein the cannabinoid component or salt thereof is entrapped in a gel matrix. In another embodiment, the gel compositions may comprise an oil-in-water (o/w) emulsion.

In some embodiments, the cosmetic composition is formulated for slow release of cannabinoid acid ester. In another embodiment, the cosmetic composition further comprises a release retarding agent or a mixture of release retarding agents. In another embodiment, the cosmetic composition is at least partly coated by an enteric-coating agent.

In some embodiments of the cosmetic composition, the composition further comprises emollient-based cream, keratolytic agent, coal tar ointment, steroid, vitamin D analog, anthralin, retinoid tazarotene, or a combination thereof.

In another embodiment, the keratolytic agent is formulated with urea or salicylic acid.

In some embodiments, the cosmetic composition further a viscosity agent. In some embodiments, the viscosity agent is a polysaccharide, a polysaccharide salt, or a combination thereof. In another embodiment, the polysaccharide is selected from hyaluronic acid (HA), chitosan, cellulose derivative, chondroitin sulfate, keratan, heparin, xanthans, galactomann, alginates, and a combination thereof.

In some embodiments, the viscosity agent is present in the composition at a concentration in the range of 1 mg/ml to 100 mg/ml. In another embodiment, the viscosity agent is present in the composition at a concentration in the range of 10 mg/ml to 25 mg/ml.

In some embodiments, the viscosity of the cosmetic composition is up to 2,000 centipoises at 20° C. In another embodiment, the viscosity of the cosmetic composition is up to 2,000 centipoises at 20° C. In another embodiment, the viscosity of the cosmetic composition is up to 1,800 centipoises at 20° C. In another embodiment, the viscosity of the cosmetic composition is up to 1,600 centipoises at 20° C. In another embodiment, the viscosity of the cosmetic composition is up to 1,500 centipoises at 20° C. In another embodiment, the viscosity of the cosmetic composition is up to 2,000 centipoises at 20° C. In another embodiment, the viscosity of the cosmetic composition is up to 2,000 centipoises at 20° C.

In some embodiments, the viscosity of the cosmetic composition is less than 5,000 centipoises between 4° C. and 12° C. In another embodiment, the viscosity of the cosmetic composition is less than 500 centipoises between 4° C. and 12° C.

In some embodiments, the viscosity of the cosmetic composition is more than 500,000 centipoises at 37° C. In another embodiment, the viscosity of the cosmetic composition is more than 1×10³ centipoises at 37° C. In another embodiment, the viscosity of the cosmetic composition is more than 3.5×10³ at 37° C.

In some embodiments, the cosmetic composition comprises at least one physiologically acceptable film forming agent. In some embodiments, the physiologically acceptable film forming agent is selected from pullulan, methyl cellulose, ethyl cellulose, sodium carboxymethyl cellulose, hydroxypropylmethyl cellulose, hydroxyethyl cellulose, hydroxypropyl cellulose, carboxymethyl cellulose, polyvinyl pyrrolidone, methacrylic acid polymers, methacrylic acid copolymers, acrylic acid polymers, acrylic acid copolymers, polyacrylamides, polyalkylene oxides, carrageenan, polyvinyl alcohol, sodium alginate, polyethylene glycol, polyacrylic acid, glycolide, polylactide, methylmethacrylate copolymer, carboxyvinyl polymer, amylose, high amylose starch, hydroxypropylated high amylose starch, alginic acid, pea starch, dextrin, pectin, chitin, chitosan, levan, elsinan and mixtures thereof. Secondary film forming agents may be added to the formulation to optimize wafer characteristics such as tensile strength, stability, flexibility and brittleness including agents such xanthan gum, tragacanth gum, guar gum, locust bean gum, acacia gum, arabic gum, collagen, gelatin, zein, gluten, soy protein isolate, whey protein isolate, casein and a mixture thereof.

In some embodiments, the cosmetic composition further comprises a cosmetically active ingredient selected from the group consisting of sunscreen agents, anti-wrinkle and anti-aging agents, skin peel agents, whitening and bleaching agents, sunless tanning agents, vitamins, skin conditioning agents, and combinations thereof.

In some embodiments, the cosmetic composition comprises 10% (w/w) or less of the tetrahydrocannabinolic acid ester compound of formula (I). In another embodiment, the cosmetic composition comprises 7% (w/w) or less of the tetrahydrocannabinolic acid ester compound of formula (I). In another embodiment, the cosmetic composition comprises 5% (w/w) or less of the tetrahydrocannabinolic acid ester compound of formula (I). In another embodiment, the cosmetic composition comprises 1% (w/w) or less of the tetrahydrocannabinolic acid ester compound of formula (I). In another embodiment, the cosmetic composition comprises 0.5% (w/w/) or less of the tetrahydrocannabinolic acid ester compound of formula (I). In another embodiment, the cosmetic composition comprises 0.1% (w/w) or less of tetrahydrocannabinolic acid ester compound of formula (I). In another embodiment, the cosmetic composition comprises 0.01% (w/w) or less of tetrahydrocannabinolic acid ester compound of formula (I). In another embodiment, the cosmetic composition comprises 0.001% (w/w) or less of tetrahydrocannabinolic acid ester compound of formula (I).

In some embodiments, the cosmetic composition comprises 10% (w/w) or less of the cannabigerolic acid ester compound of formula (II). In another embodiment, the cosmetic composition comprises 7% (w/w) or less of the cannabigerolic acid ester compound of formula (II). In another embodiment, the cosmetic composition comprises 5% (w/w) or less of the cannabigerolic acid ester compound of formula (II). In another embodiment, the cosmetic composition comprises 1% (w/w) or less of the cannabigerolic acid ester compound of formula (II). In another embodiment, the cosmetic composition comprises 0.5% (w/w/) or less of the cannabigerolic acid ester compound of formula (II). In another embodiment, the cosmetic composition comprises 0.1% (w/w) or less of cannabigerolic acid ester compound of formula (II). In another embodiment, the cosmetic composition comprises 0.01% (w/w) or less of cannabigerolic acid ester compound of formula (II). In another embodiment, the cosmetic composition comprises 0.001% (w/w) or less of cannabigerolic acid ester compound of formula (II).

In some embodiments, the cosmetic composition comprises 10% (w/w) or less of the cannabinolic acid ester compound of formula (III). In another embodiment, the cosmetic composition comprises 7% (w/w) or less of the cannabinolic acid ester compound of formula (III). In another embodiment, the cosmetic composition comprises 5% (w/w) or less of the cannabinolic acid ester compound of formula (III). In another embodiment, the cosmetic composition comprises 1% (w/w) or less of the cannabinolic acid ester compound of formula (III). In another embodiment, the cosmetic composition comprises 0.5% (w/w/) or less of the cannabinolic acid ester compound of formula (III) In another embodiment, the cosmetic composition comprises 0.1% (w/w) or less of cannabinolic acid ester compound of formula (III). In another embodiment, the cosmetic composition comprises 0.01% (w/w) or less of cannabinolic acid ester compound of formula (III). In another embodiment, the cosmetic composition comprises 0.001% (w/w) or less of cannabinolic acid ester compound of formula (III).

### Edible Compositions

The present disclosure also provides edible compositions including a cannabinoid acid ester compound (e.g., as described herein, such as a compound of any one of formulae (I)-(IX)), and a physiologically acceptable excipient. The subject edible compositions find use in a variety of therapeutic indications and other uses (e.g., as described herein).

Accordingly, the present disclosure also provides an edible composition comprising:

a tetrahydrocannabinolic acid (THCA) ester compound of formula (I);

a cannabigerolic acid (CBGA) ester compound of formula (II);

a cannabinolic acid (CBNA) ester compound of formula (III); or

a combination thereof;

wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a physiologically acceptable salt thereof, and a physiologically acceptable excipient.

In some embodiments of the composition, R₁ is C₁-C₆ alkyl or substituted C₁-C₆ alkyl. In some embodiments of the composition, R₁ is selected from methyl, ethyl, propyl, butyl, and pentyl. In some embodiments of the composition, R₁ is methyl.

In some embodiments of the composition, R₂ is C₁-C₁₀ alkyl, or substituted C₁-C₁₀ alkyl. In some embodiments of the composition, R₂ is C₂₋₆ alkyl. In some embodiments of the composition, R₂ is pentyl.

In some embodiments, the THCA ester compound of formula (I) is a THCA ester compound of formula (Ia), or a pharmaceutically acceptable salt thereof.

In some embodiments, the THCA ester compound of formula (I) is a THCA ester compound of formula (Ib), or a pharmaceutically acceptable salt thereof.

In some embodiments of the edible composition, the THCA-Me compound is selected from an enantiomerically pure THCA-Me, a stereoisomeric mixture of THCA-Me, a diastereomeric mixture of THCA-Me, a salt thereof, a deuterated analog thereof, a fluorinated analog thereof, and a combination thereof.

In some embodiments, the THCA ester compound of formula (I) is the Δ⁹-tetrahydrocannabinolic acid methyl ester (Δ⁹-THCA-Me) compound of formula (Ic), or a physiologically acceptable salt thereof.

In some embodiments, the THCA ester compound of formula (I) is the Δ⁸-tetrahydrocannabinolic acid methyl ester (Δ⁸-THCA-Me) compound of formula (Id), or a physiologically acceptable salt thereof.

In some embodiments, the CBGA ester compound of formula (II) is the cannabigerolic acid methyl ester (CBGA-Me) compound of formula (IIa), or a physiologically acceptable salt thereof.

In some embodiments of the edible composition, the CBGA-Me compound is selected from a diastereomeric mixture of CBGA-Me, a salt thereof, a deuterated analog thereof, a fluorinated analog thereof, and a combination thereof.

In some embodiments, the CBNA ester compound of formula (III) is the cannabinolic acid methyl ester (CBNA-Me) compound of formula (IIIa), or a physiologically acceptable salt thereof.

According to some embodiments, the physiologically acceptable excipient is an aqueous solution or carrier. In some embodiments, the aqueous solution is a buffer having physiological or near-physiological pH, such as phosphate buffered saline (PBS). In some embodiments, the physiologically acceptable excipient is selected from emulsifiers, buffering agents, pH adjusting agents, tonicity modifiers, preservatives, antioxidants, stabilizers, and a combination thereof. Exemplary excipients, additives and additional components of the subject physiological compositions are described in further detail below.

In some embodiments, the subject edible composition can further include one or more additional cannabinoid compounds. The additional cannabinoid compound can be an isolated compound, or part of a complex mixture. The additional cannabinoid compound can be part of a crude component or composition ingredient, or a purified sample.

In some embodiments, the one or more additional cannabinoid compounds that are included into a cosmetic composition are independently comprised in one or more Cannabis plant extracts. The Cannabis plant extracts can be obtained from any convenient source. In some embodiments, the Cannabis plant extracts are produced from a plant strain selected from Cannabis sativa, Cannabis indica, Cannabis ruderalis, a hybrid strain, a strain with a high concentration of cannabidiol (CBD), a strain with a high concentration of tetrahydrocannabinol (THC), and a combination thereof.

In some embodiments, the one or more Cannabis plant extracts comprise a cannabinoid compound selected from cannabidiol (CBD), tetrahydrocannabinol (THC), cannabinol (CBN), cannabigerol (CBG), cannabichromene (CBC), acids thereof, esters of the acids thereof, and combinations thereof.

In some embodiments, the one or more additional cannabinoid compounds are independently selected from cannabidiol (CBD), cannbigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C₃ (THC-C₃), tetrahydrocannabinol-C₄ (THC-C₄), tetrahydrocannabinol-C₇ (THC-C₇), and esters thereof, stereoisomers thereof, deuterated analogs thereof, fluorinated analogs thereof, and combinations thereof.

In some embodiments, the one or more Cannabis plant extracts that are incorporated into a cosmetic composition include about 1% (w/w) or more of CBD, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more CBD. In certain embodiments, the one or more Cannabis plant extracts comprise about 30% (w/w) or less of CBD.

In some embodiments, the one or more Cannabis plant extracts comprise about 1% (w/w) or more of THC, such as about 2% (w/w) or more, about 3% (w/w) or more, about 4% (w/w) or more, about 5% (w/w) or more, about 6% (w/w) or more, about 7% (w/w) or more, about 8% (w/w) or more, about 9% (w/w) or more, about 10% (w/w), about 15% (w/w), about 20% (w/w) or more, or about 25% (w/w) or more THC. In certain embodiments, the one or more Cannabis plant extracts comprise about 30% (w/w) or less of THC.

In some embodiments, the one or more Cannabis plant extracts comprising the one or more additional cannabinoid compounds are produced by extraction from a Cannabis plant with a suitable solvent, or a combination of solvents. In some embodiments, the solvent is a physiologically acceptable solvent, for example but not limited to, a non-toxic solvent that is suitable for administration to a mammal with no unacceptable adverse effects. The solvent may be an aqueous or non-aqueous solvent.

In some embodiments, the solvent for extraction is a polar solvent, a hydrocarbon solvent, an alcohol solvent, carbon dioxide, an oil, or a combination thereof.

In some embodiments, the solvent for extraction is an oil comprising copaiba oil, vegetable oil, olive oil, sesame oil, coconut oil, avocado oil, peanut oil, canola oil, cottonseed oil, soybean oil, safflower oil, sunflower oil, castor oil, corn oil, palm oil, poppy seed oil, or walnut oil. In another embodiment, the cosmetic composition comprises copaiba oil.

In some embodiments, the edible composition comprises 1% to 20% (w/w) a cannabinoid acid ester compound and 50% to 90% vegetable oil.

In some embodiments, the edible composition comprises 1% to 10% (w/w) of the tetrahydrocannabinolic acid ester compound of formula (I).

In some embodiments, the THCA ester compound is the Δ⁹-THCA-Me ester compound of formula (Ic). In another embodiment, the edible composition comprises 1% to 10% Δ⁹-THCA-Me, 20% to 60% alcohol, and 0% to 35% propylene glycol. In another embodiment, the edible composition comprises 2% to 10% Δ⁹-THCA-Me, 30% to 60% ethanol, and a combination of propylene glycol and polyethylene glycol. In another embodiment, the edible composition further comprises 5% to 20% polyethylene glycol.

In some embodiments, the THCA ester compound is the Δ⁸-THCA-Me ester compound of formula (Id). In another embodiment, the edible composition comprises 1% to 10% Δ⁸-THCA-Me, 20% to 60% alcohol, and 0% to 35% propylene glycol. In another embodiment, the edible composition comprises 2% to 10% Δ⁸-THCA-Me, 30% to 60% ethanol, and a combination of propylene glycol and polyethylene glycol. In another embodiment, the edible composition further comprises 5% to 20% polyethylene glycol.

In some embodiments, the ratio of the THCA ester compound of formula (I) to additional cannabinoid compound in the composition is from 1.05:1 to 1,000:1.

In some embodiments, the edible composition comprises 1% to 10% (w/w) of the cannabigerolic acid (CBGA) ester compound of formula (II).

In some embodiments, the CBGA ester compound is the CBGA-Me ester compound of formula (IIa). In another embodiment, the edible composition comprises 1% to 10% CBGA-Me, 20% to 60% alcohol, and 0% to 35% propylene glycol. In another embodiment, the edible composition comprises 2% to 10% CBGA-Me, 30% to 60% ethanol, and a combination of propylene glycol and polyethylene glycol. In another embodiment, the edible composition further comprises 5% to 20% polyethylene glycol.

In another embodiment, the ratio of the CBGA ester compound of formula (II) to additional cannabinoid compounds in the composition is from 1.05:1 to 1,000:1.

In some embodiments, the edible composition comprises 1% to 10% (w/w) of the cannabinolic acid (CBNA) ester compound of formula (III).

In some embodiments, the CBNA ester compound is the CBNA-Me ester compound of formula (IIIa). In another embodiment, the edible composition comprises 1% to 10% CBNA-Me, 20% to 60% alcohol, and 0% to 35% propylene glycol. In another embodiment, the edible composition comprises 2% to 10% CBNA-Me, 30% to 60% ethanol, and a combination of propylene glycol and polyethylene glycol. In another embodiment, the edible composition further comprises 5% to 20% polyethylene glycol.

In some embodiments, the ratio of the CBNA ester compound of formula (III) to additional cannabinoid compounds in the composition is from 1.05:1 to 1,000:1.

In some embodiments, the edible composition further comprises a lipid. The lipid can be triglyceride, fat, oil, fatty acid, or a mixture thereof.

In some embodiments, the edible composition further comprises triglyceride, fat, oil, fatty acid, or a mixture thereof. In some embodiments of the pharmaceutical composition, the composition further comprises cyclodextrin, electrolyte, vitamin, mineral, flavoring agent, or a combination thereof.

In some embodiments, the edible composition further comprises cyclodextrin. In another embodiment, the cyclodextrin is selected from hydroxypropyl β-cyclodextrin, sulfobutylether β-cyclodextrin, and methyl β-cyclodextrin (MPCD).

In some embodiments, the physiologically acceptable excipient is aqueous. In another embodiment, the physiologically acceptable excipient is selected from an emulsifier, a buffering agent, a pH adjusting agent, a preservative, an antioxidant, a stabilizer, an electrolyte, a vitamin, a mineral, a flavoring agent, a solubilizing agent, a tonicity enhancing agent, a colorant, and a combination thereof.

In some embodiments, the colorant comprises alumina (dried aluminum hydroxide), annatto extract, calcium carbonate, canthaxanthin, caramel, β-carotene, cochineal extract, carmine, potassium sodium copper chlorophyllin (chlorophyllin copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

In some embodiments, the pH-adjusting agent is an organic or mineral acid.

In some embodiment of the edible composition, the composition is formulated for administration orally.

In some embodiment, the edible composition is in a form selected from liquid, gel, cream, ointment, lotion, paste, tablet, pill, capsule, pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, and suppository.

In some embodiment, the edible composition is formulated at a temperature in the range of 4° C. to 37° C.

In some embodiments, the edible composition is formulated in the form of a gel at physiological temperature. In another embodiment, the edible composition is a gel, wherein the cannabinoid component or salt thereof is entrapped in a gel matrix. In another embodiment, the gel compositions may comprise an oil-in-water (o/w) emulsion.

In some embodiments, the edible composition is formulated for slow release of cannabinoid acid ester. In another embodiment, the edible composition further comprises a release retarding agent or a mixture of release retarding agents. In another embodiment, the edible composition is at least partly coated by an enteric-coating agent.

In some embodiments of the edible composition, the composition further comprises emollient-based cream, keratolytic agent, coal tar ointment, steroid, vitamin D analog, anthralin, retinoid tazarotene, or a combination thereof.

In another embodiment, the keratolytic agent is formulated with urea or salicylic acid.

In some embodiments, the edible composition further a viscosity agent. In some embodiments, the viscosity agent is a polysaccharide, a polysaccharide salt, or a combination thereof. In another embodiment, the polysaccharide is selected from hyaluronic acid (HA), chitosan, cellulose derivative, chondroitin sulfate, keratan, heparin, xanthans, galactomann, alginates, and a combination thereof.

In some embodiments, the viscosity agent is present in the composition at a concentration in the range of 1 mg/ml to 100 mg/ml. In another embodiment, the viscosity agent is present in the composition at a concentration in the range of 10 mg/ml to 25 mg/ml.

In some embodiments, the viscosity of the edible composition is up to 2,000 centipoises at 20° C. In another embodiment, the viscosity of the edible composition is up to 2,000 centipoises at 20° C. In another embodiment, the viscosity of the edible composition is up to 1,800 centipoises at 20° C. In another embodiment, the viscosity of the edible composition is up to 1,600 centipoises at 20° C. In another embodiment, the viscosity of the edible composition is up to 1,500 centipoises at 20° C. In another embodiment, the viscosity of the edible composition is up to 2,000 centipoises at 20° C. In another embodiment, the viscosity of the edible composition is up to 2,000 centipoises at 20° C.

In some embodiments, the viscosity of the edible composition is less than 5,000 centipoises between 4° C. and 12° C. In another embodiment, the viscosity of the edible composition is less than 500 centipoises between 4° C. and 12° C.

In some embodiments, the viscosity of the edible composition is more than 500,000 centipoises at 37° C. In another embodiment, the viscosity of the edible composition is more than 1×10³ centipoises at 37° C. In another embodiment, the viscosity of the edible composition is more than 3.5×10³ at 37° C.

In some embodiments, the edible composition comprises at least one physiologically acceptable film forming agent. In some embodiments, the physiologically acceptable film forming agent is selected from pullulan, methyl cellulose, ethyl cellulose, sodium carboxymethyl cellulose, hydroxypropylmethyl cellulose, hydroxyethyl cellulose, hydroxypropyl cellulose, carboxymethyl cellulose, polyvinyl pyrrolidone, methacrylic acid polymers, methacrylic acid copolymers, acrylic acid polymers, acrylic acid copolymers, polyacrylamides, polyalkylene oxides, carrageenan, polyvinyl alcohol, sodium alginate, polyethylene glycol, polyacrylic acid, glycolide, polylactide, methylmethacrylate copolymer, carboxyvinyl polymer, amylose, high amylose starch, hydroxypropylated high amylose starch, alginic acid, pea starch, dextrin, pectin, chitin, chitosan, levan, elsinan and mixtures thereof. Secondary film forming agents may be added to the formulation to optimize wafer characteristics such as tensile strength, stability, flexibility and brittleness including agents such xanthan gum, tragacanth gum, guar gum, locust bean gum, acacia gum, arabic gum, collagen, gelatin, zein, gluten, soy protein isolate, whey protein isolate, casein and a mixture thereof.

In some embodiments, the edible composition further comprises edible excipient selected from the group consisting of fillers, emulsifiers, anticaking agents, preservatives, colorants, flavoring agents, electrolytes, and combinations thereof.

In some embodiments, the emulsifier is selected from polyvinyl alcohol (PVA), polysorbate, polyethylene glycols, polyoxyethylene-polyoxypropylene block copolymers, polyglycerin fatty acid esters, sorbitan fatty acid ester, polyoxyethylen sorbitan fatty acid ester, polyethylene glycol fatty acid ester, polyoxyethylene alkyl ether, polyoxyethylene castor oil, hardened castor oil, hydrogenated castor oil, sodium gluconate, acrylates, C₁₀₋₃₀ alkyl acrylate crosspolymer, sodium carboxymethyl betaglucan, polyglyceryl-3 methylglucose distearate, cetearyl alcohol, cetyl alcohol, stearic acid, behenyl alcohol, butylene glycol, propylene glycol, xanthan gum, potassium cetyl phosphate, polyglyceryl-6 distearate jojoba esters, polyglyceryl-3-beeswax, PEG-800, laureth-7, C₁₃₋₁₄ isoparaffin, polyisobutene, PEG-200 hydrogenated glyceryl palmate, cellulose gum, PEG-7 glyceryl cocoate, aluminum starch octenylsuccinate, and a combination thereof.

In some embodiments, the anticaking agent is selected from the group consisting of cellulose, microcrystalline cellulose, silicon dioxide, tri-calcium phosphate, sodium chloride, sodium bicarbonate, sodium aluminum silicate, magnesium stearate, magnesium carbonate, and combinations thereof.

In some embodiments, the preservative is selected from the group consisting of sulfur dioxide and sulfites, sorbic acid, sodium sorbate, calcium sorbate, potassium sorbate, benzoic acid, sodium benzoate, potassium benzoate, lactic acid, propionic acid, sodium propionate, and combinations thereof.

In some embodiments, the colorant is selected from the group consisting of brilliant blue (E133), indigotine (E132), fast green (E143), erythrosine (E127), allura red (E129), tartrazine (E102), sunset yellow (E110), and natural food dyes.

In some embodiments, the natural food dye is selected from the group consisting of annatto (E160b), caramel (E150a-d), carmine (E120), dactylopius coccus, elderberry juice (E163), lycopene (E160d), paprika (E160c), turmeric (E100), and combinations thereof.

In some embodiments, the flavoring agent is extracted from apple, cherry, green tea, cinnamon, clove, black tea, plum, mango, date, watermelon, coconut, pear, jasmine, peach, fennel, melon, lychee, mint, chocolate, coffee, cream, banana, almond, grape, strawberry, blueberry, blackberry, pine, kiwi, sapote, taro, lotus, pineapple, orange, lemon, licorice, vanilla, rose, osmanthus, ginseng, spearmint, citrus, cucumber, honeydew, walnut, honey, or any combination thereof.

In some embodiments, the edible composition further comprises a nutrient selected from the group consisting of vitamins, minerals, and combinations thereof.

In some embodiments, the vitamin is selected from the group consisting of vitamin A (retinols and carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), vitamin K (quinones), and combinations thereof.

In some embodiments, the mineral is selected from the group consisting of magnesium, calcium, iron, zinc, chrome, selenium, potassium, silicon, and combinations thereof.

In some embodiments, the edible composition is in a form selected from the group consisting of powder, flake, granular, capsule, tablet, syrup, solution, emulsion and suspension.

In some embodiments, the edible composition is a food product selected from bread, cereals, pasta, pastry, biscuits, candy, and confectionary.

In some embodiments, the edible composition is a beverage.

In some embodiments, the edible composition has beneficial effect on the microbiome of the subject.

In some embodiments, the edible composition comprises 10% (w/w) or less of the tetrahydrocannabinolic acid ester compound of formula (I). In another embodiment, the edible composition comprises 7% (w/w) or less of the tetrahydrocannabinolic acid ester compound of formula (I). In another embodiment, the edible composition comprises 5% (w/w) or less of the tetrahydrocannabinolic acid ester compound of formula (I). In another embodiment, the edible composition comprises 1% (w/w) or less of the tetrahydrocannabinolic acid ester compound of formula (I). In another embodiment, the edible composition comprises 0.5% (w/w/) or less of the tetrahydrocannabinolic acid ester compound of formula (I). In another embodiment, the edible composition comprises 0.1% (w/w) or less of tetrahydrocannabinolic acid ester compound of formula (I). In another embodiment, the edible composition comprises 0.01% (w/w) or less of tetrahydrocannabinolic acid ester compound of formula (I). In another embodiment, the edible composition comprises 0.001% (w/w) or less of tetrahydrocannabinolic acid ester compound of formula (I).

In some embodiments, the edible composition comprises 10% (w/w) or less of the cannabigerolic acid ester compound of formula (II). In another embodiment, the edible composition comprises 7% (w/w) or less of the cannabigerolic acid ester compound of formula (II). In another embodiment, the edible composition comprises 5% (w/w) or less of the cannabigerolic acid ester compound of formula (II). In another embodiment, the edible composition comprises 1% (w/w) or less of the cannabigerolic acid ester compound of formula (II). In another embodiment, the edible composition comprises 0.5% (w/w/) or less of the cannabigerolic acid ester compound of formula (II). In another embodiment, the edible composition comprises 0.1% (w/w) or less of cannabigerolic acid ester compound of formula (II). In another embodiment, the edible composition comprises 0.01% (w/w) or less of cannabigerolic acid ester compound of formula (II). In another embodiment, the edible composition comprises 0.001% (w/w) or less of cannabigerolic acid ester compound of formula (II).

In some embodiments, the edible composition comprises 10% (w/w) or less of the cannabinolic acid ester compound of formula (III). In another embodiment, the edible composition comprises 7% (w/w) or less of the cannabinolic acid ester compound of formula (III). In another embodiment, the edible composition comprises 5% (w/w) or less of the cannabinolic acid ester compound of formula (III). In another embodiment, the edible composition comprises 1% (w/w) or less of the cannabinolic acid ester compound of formula (III). In another embodiment, the edible composition comprises 0.5% (w/w/) or less of the cannabinolic acid ester compound of formula (III) In another embodiment, the edible composition comprises 0.1% (w/w) or less of cannabinolic acid ester compound of formula (III). In another embodiment, the edible composition comprises 0.01% (w/w) or less of cannabinolic acid ester compound of formula (III). In another embodiment, the edible composition comprises 0.001% (w/w) or less of cannabinolic acid ester compound of formula (III).

### Methods of Use

Aspects of the present disclosure include methods of improving the appearance, the well-being, and/or a health condition in a subject (e.g., as described herein) that include administration of a subject cannabinoid acid ester compound (e.g., as described herein) or subject cosmetic composition (e.g., as described herein), or subject edible composition (e.g., as described herein) to a subject in need thereof.

In some embodiments, the cosmetic composition is administered topically or transdermally. In certain embodiments, the cosmetic composition is selected from make-up product, foundation products, and skin-care products.

In some embodiments, the edible composition id administered orally. In certain embodiments, the edible composition is selected from food products, nutraceuticals, beverages, and animal feed.

### Methods of Preparation

The compositions of the present disclosure may be formulated as single-phase aqueous, emulsion or multiple emulsions. According to some embodiments, the composition is formulated as emulsion. These emulsions may be oil-in-water (o/w) (including silicone in water) emulsions, water-in-oil (including water-in-silicone) (w/o) emulsions, or multiple emulsions such as oil-in-water-in-oil (o/w/o) or water-in-oil-in-water (w/o/w). It is understood that the oil phase can comprise silicone oils, non-silicone organic oils, or mixtures thereof. The compositions can comprise two immiscible phases that are admixed at the time of use by shaking. Each possibility represents a separate embodiment of the present disclosure.

According to some embodiments, the composition is made by preparing a dispersion of each component in a suitable solvent (dispersant), adjusting the dispersion pH with a pH adjusting agent, if necessary, and admixing the dispersions with shear to permit the formation of the desired matrix.

A common mode of administration of medical Cannabis is by dissolving the Cannabis extract or pure cannabinoid in triglyceride oils, such as vegetable oils, for oral delivery and administration. The oil is either filled into capsules or used as-in in various volumes. In contrast to administration by inhalation, the oral route of administration is perceived as an acceptable mode of self-medication, such as consuming a pill, a tablet, or a capsule. In such cases, an immediate release of the cannabinoid is obtained with fast absorption and an intermediate duration time of activity, but longer than smoking or vaporization.

A person of ordinary skill in the art can select the appropriate presentation or administration form, and the method of preparing it on the basis of general knowledge, taking into account the nature of the constituents used and the intended use of the composition.

The present disclosure also provides for kits comprising the above compositions. The compositions as described herein can be packaged to comprise, either separately or in the kit form together with a container, instructions for using or instruction brochures for using the compositions as described herein.

### Combination Therapy

The compositions of the present disclosure may be used in pharmaceutical combinations with other therapeutic agents that may also be useful for treating the diseases or disorders as described herein. Such therapeutic agents may be administered by a route, in a therapeutically effective amount as commonly used thereof, contemporaneously or sequentially with the compositions as described herein.

The compositions as described in the present disclosure may be used contemporaneously with one or more other therapeutic agents in a unit dosage form. The combination therapy described herein may also comprise therapies in which the composition as described herein and the one or more other therapeutic agents are administered concomitantly, wherein the one or more other therapeutic agents are administered on the same or different schedules as the composition as described herein.

When oral formulations are used, the composition described herein and the other therapeutic agents may be combined into a single combination tablet or other oral dosage form, or the other therapeutic agents may be packaged together as separate tablets or other oral dosage forms. When used in combination with one or more other active ingredients, the active ingredients may be used in lower doses than when each is used alone.

In some embodiments, the pharmaceutical composition as described herein is used in combination with one or more additional therapeutic agents for treating various diseases and disorders as described herein.

In some embodiments, the pharmaceutical composition as described herein and the one or more additional therapeutic agents is administered simultaneously, concurrently, alternately, sequentially, or successively. In another embodiment, the pharmaceutical composition as described herein and the one or more additional therapeutic agents are administered according to an overlapping schedule.

In some embodiments, the pharmaceutical composition described herein may be administered at any point prior to another treatment, wherein the time prior another treatment is 150 hr, 145 hr, 140 hr, 135 hr, 130 hr, 125 hr, 120 hr, 115 hr, 110 hr, 105 hr, 100 hr, 95 hr, 90 hr, 85 hr, 80 hr, 75, 70 hr, 65 hr, 60 hr, 55 hr, 50 hr, 45 hr, 40 hr, 35 hr, 30 hr, 25 hr, 20 hr, 15 hr, 10 hr, 5 hr, 4 hr, 3 hr, 2 hr, 1 hr, 50 mins., 40 mins., 30 mins., 20 mins., 15 mins, 10 mins, 9 mins, 8 mins, 7 mins., 6 mins., 5 mins., 4 mins., 3 mins, 2 mins, or 1 min prior to the other treatment.

In some embodiments, the pharmaceutical composition described herein may be administered at any point prior to a second treatment of the pharmaceutical composition as described herein, wherein the time prior to the second treatment is about 150 hr, 145 hr, 140 hr, 135 hr, 130 hr, 125 hr, 120 hr, 115 hr, 110 hr, 105 hr, 100 hr, 95 hr, 90 hr, 85 hr, 80 hr, 75, 70 hr, 65 hr, 60 hr, 55 hr, 50 hr, 45 hr, 40 hr, 35 hr, 30 hr, 25 hr, 20 hr, 15 hr, 10 hr, 5 hr, 4 hr, 3 hr, 2 hr, 1 hr, 50 mins., 40 mins., 30 mins., 20 mins., 15 mins, 10 mins, 9 mins, 8 mins, 7 mins., 6 mins., 5 mins., 4 mins., 3 mins, 2 mins, or 1 min.

In some embodiments, the route of administration of the pharmaceutical composition as described herein or the one or more additional therapeutic agents can be by any route and will be determined based on the physician and the subject on an individual basis. All other routes of administration of a therapeutically effective amount of an agent or the pharmaceutical composition as described herein are included by the present disclosure.

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

As used herein, the symbol “ [symbol] ” refers to a covalent bond comprising a single or a double bond.

The term “alkyl” refers to an unbranched or branched saturated hydrocarbon chain. In some embodiments, alkyl as used herein has 1 to 50 carbon atoms ((C₁-C₅₀)alkyl), 1 to 20 carbon atoms ((C₁-C₂₀)alkyl), 1 to 10 carbon atoms ((C₁-C₁₀)alkyl), 1 to 8 carbon atoms ((C₁-C₈)alkyl), 1 to 6 carbon atoms ((C₁-C₆)alkyl), or 1 to 4 carbon atoms ((C₁-C₄)alkyl). Examples of alkyl groups may, for example, include methyl, ethyl, n-propyl, isopropyl, n-butyl, sec-butyl, tert-butyl, n-pentyl, 2-pentyl, isopentyl, neopentyl, n-hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “substituted alkyl” refers to an alkyl group as defined herein wherein one or more carbon atoms in the alkyl chain have been optionally replaced with a heteroatom such as O—, N—, S—, —S(O)n- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkylene” refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkenyl” refers to an aliphatic hydrocarbon group containing at least one carbon-carbon double bond including straight-chain, branched-chain and cyclic alkenyl groups. In some embodiments, the alkenyl group has 2-10 carbon atoms (a C₂₋₁₀ alkenyl). In another embodiment, the alkenyl group has 2-4 carbon atoms in the chain (a C₂₋₄ alkenyl). Exemplary alkenyl groups include, but are not limited to, ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl

The term “alkynyl” refers to straight or branched monovalent hydrocarbyl groups having from 2 to 6 carbon atoms and preferably 2 to 3 carbon atoms and having at least 1 and preferably from 1 to 2 sites of acetylenic (C≡C—) unsaturation. Examples of such alkynyl groups include, but are not limited to, acetylenyl (C≡CH), and propargyl (CH₂C≡CH).

The terms “alkenylene,” “alkynylene,” “arylene,” “arylalkylene,” and “alkylarylene” refer to di-radical alkenyl, alkynyl, aryl, arylalkyl, and alkylaryl groups, respectively.

The terms “cycloalkyl” and “bicycloalkyl” refers to cyclic alkyl groups of from 3 to 10 carbon atoms having single or multiple cyclic rings including fused, bridged, and spiro ring systems. Examples of suitable cycloalkyl groups include, but are not limited to, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The terms “substituted cycloalkyl” and “substituted bicycloalkyl” refer to cycloalkyl groups having from 1 to 5 substituents, or from 1 to 3 substituents. selected from alkyl, substituted alkyl, alkoxy, substituted alkoxy, cycloalkyl, substituted cycloalkyl, bicycloalkyl, substituted bicycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted” refers that any one or more hydrogen atoms on the designated atom or group is replaced with one or more substituents other than hydrogen, provided that the designated atom's normal bonding valence is not exceeded. The one or more substituents include, but are not limited to, alkyl alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocycloalkyl, hydroxy, hydrazino, imino, oxo, nitro, alky sulfinyl, sulfonic acid, alkylsulfonyl, thiocyanate, thiol, thione, or combinations thereof. By way of example, there may be one, two, three, four, five, or six substituents.

The phrase “optionally substituted” means that a non-hydrogen substituent may or may not be present on a given atom, and, thus, the description includes structures wherein a non-hydrogen substituent is present and structures wherein a non-hydrogen substituent is not present.

The phrases “of the formula” and “of the structure” are not intended to be limiting and are used in the same way that the term “comprising” is commonly used. The term “independently selected from” is used herein to indicate that the recited elements, i.e., R groups or the like, can be identical or different.

The term “isomers” refers to two or more compounds comprising the same numbers and types of atoms, groups or components, but with different structural arrangement and connectivity of the atoms.

The term “tautomer” refers to one of two or more structural isomers which readily convert from one isomeric form to another and which exist in equilibrium.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposeable mirror images of one another.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures also can be resolved into their respective enantiomers by well-known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. See, for example, Carreira and Kvaerno, Classics in Stereoselective Synthesis, Wiley-VCH: Weinheim, 2009.

Geometric isomers, resulting from the arrangement of substituents around a carbon-carbon double bond or arrangement of substituents around a cycloalkyl or heterocyclic ring, can also exist in the compounds of the present disclosure. The symbol=denotes a bond that may be a single, double or triple bond as described herein. Substituents around a carbon-carbon double bond are designated as being in the “Z” or “E” configuration, where the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituent on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compound wherein the substituents are disposed on both the same and opposite sides of the plane of the ring are designated “cis/trans.”

The present disclosure also encompasses isotopically labeled compounds which are identical to those compounds recited herein, except that one or more atoms are replaced by an atom having an atomic mass or mass number different from the atomic mass or mass number usually found in nature (“isotopologues”). Examples of isotopes that can be incorporated into compounds described herein include isotopes of hydrogen, carbon, nitrogen, oxygen, phosphorus, fluorine and chlorine, such as ²H (“D”), ³H, ¹³C, ¹⁴C, ¹⁵N, ¹⁸O, ¹⁷O, ³¹P, ³²P, ³⁵S, ¹⁸F, and ³⁶Cl, respectively. For example, a compound described herein can have one or more H atoms replaced with deuterium.

Certain isotopically labeled compounds, such as those labeled with ³H and ¹⁴C, can be useful in compound and/or substrate tissue distribution assays. Tritiated (³H) and carbon-14 (¹⁴C) isotopes can be particularly preferred for their ease of preparation and detectability. Further, substitution with heavier isotopes such as deuterium can afford certain therapeutic advantages resulting from greater metabolic stability, such as increased in vivo half-life or reduced dosage requirements, and hence can be preferred in some circumstances. Isotopically labeled compounds can generally be prepared by following procedures analogous to those disclosed herein, for example, in the Examples section, by substituting an isotopically-labeled reagent for a non-isotopically-labeled reagent.

Singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

A dash (“-”) symbol that is not between two letters or symbols refers to a point of bonding or attachment for a substituent. For example, —NH₂ is attached through the nitrogen atom.

The terms “active agent,” “drug,” “pharmacologically active agent,” and “active pharmaceutical ingredient” are used interchangeably to refer to a compound or composition which, when administered to a subject, induces a desired pharmacologic or physiologic effect by local or systemic action or both.

The terms “individual,” “host,” and “subject,” are used interchangeably, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

“Patient” refers to a human subject.

The terms “treating,” “treatment,” and the like, refer to obtaining a desired pharmacologic and/or physiologic effect, such as reduction of one or more symptoms of the disease or disorder. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (i.e., reduction in pain or other symptom).

The term “amelioration” or any grammatical variation thereof (e.g., ameliorate, ameliorating, and amelioration etc.), includes, but is not limited to, delaying the onset, or reducing the severity of a disease or condition (e.g., diarrhea, bacteremia and/or endotoxemia). Amelioration, as used herein, does not require the complete absence of symptoms.

The term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈ alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

The phrase “therapeutically effective amount” refers to the amount of a compound that, when administered to a mammal or other subject for treating a disease, condition, or disorder, is sufficient to affect such treatment for the disease, condition, or disorder. The “therapeutically effective amount” will vary depending on the compound, the disease and its severity and the age, weight, etc., of the subject to be treated.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “pharmaceutically acceptable excipient” includes both one and more than one such excipient, diluent, carrier, and/or adjuvant.

The term “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the term “sustained release”, “delayed release”, and “controlled release” refer to prolonged or extended release of the therapeutic agent or API of the pharmaceutical formulation. These terms may further refer to composition which provides prolonged or extended duration of action, such as pharmacokinetics (PK) parameters of a pharmaceutical composition comprising a therapeutically effective amount of the active pharmaceutical ingredient as described herein.

As used herein, the term “extract” as used herein refers a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence which is free of, or substantially free of solvent. The term extract may be a single extract obtained from a particular extraction step or series of extraction steps. Extract also may be a combination of extracts obtained from separate extraction steps or separate feedstocks. Such combined extracts are thus also encompassed by the term “extract”. Any methods of extraction with suitable solvent are encompassed. Exemplary extraction methods can be found for example in U.S. Pat. No. 6,403,126. The extract may be obtained from any part of the plant e.g. from leaves, flowers, stems, roots, fruits and seeds. The extract may be aqueous or oily.

As used herein, the term “Cannabis extract” refers to one or more plant extracts from the Cannabis plant. A Cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of Cannabis plant material. Such means include but are not limited to supercritical or subcritical extraction with CO₂, extraction with hot or cold gas and extraction with solvents. In some embodiments, the term refers to a mixture of liquid or semi-solid, resinous substances obtained through extraction from two or more different Cannabis species. In some embodiments, the term refers also to a compound purified from the extract.

As used herein, the term “semi-solid” refers to a form which is a capable of supporting its own weight and holds its shape or is capable of conforming to other shapes.

As used herein, the term “Cannabis plant” refers to plants of the genus Cannabis, including but not limited to Cannabis sativa, Cannabis indica, and Cannabis ruderalis. According to some embodiment, Cannabis plant is a CBD-rich strain of Cannabis plant or THC-rich strain of Cannabis plant. Each possibility represents a separate embodiment.

As used herein, “hybrid strain” refers to different strains of Cannabis which include differing amounts and/or ratios of the various cannabinoid compounds. For example, Cannabis sativa typically has a relatively high THC/CBD ratio. Conversely, Cannabis indica generally has a relatively low THC/CBD ratio compared to Cannabis sativa, although the absolute amount of THC can be higher in Cannabis indica than in Cannabis sativa.

As used herein, the terms “high-CBD strain” and “CBD-rich strain” are directed to a strain of Cannabis plant which comprises CBD and optionally one or more additional cannabinoids, such as, for example but not limited to: THC, CBN, and the life.

As used herein, the terms “high-THC strain” and “THC-rich strain” are directed to a strain of Cannabis plant which comprises THC and optionally one or more additional cannabinoids, such as, for example but not limited to: CBD, CBN, and the like.

The cannabinoid component combination of the present disclosure is generally prepared by conventional methods such as are known in the art of making a mixture in the ratio described above. Such methods typically involve mixing of the THCA, CBGA, or CBNA ester and one or more additional cannabinoid compound(s), or one or more extract of a Cannabis plant in one or more steps to a relatively uniform state, with or without heating, cooling, application of vacuum, and the like.

The above definitions are not intended to include impermissible substitution patterns (i.e., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “alkylaryl.” Unless specified otherwise, where a group is described as optionally substituted, any substituents of the group are themselves unsubstituted.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ¹⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of R-enantiomer and S-enantiomer, and enantio-enriched stereomeric mixtures comprising of R- and S-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

Unless otherwise stated, all temperatures are in degrees Celsius. Efforts have been made to ensure accuracy with respect to numbers used (e.g., amounts, temperatures, etc.), but some experimental errors and deviation should be allowed for.

All experiments conformed to the ethical guidelines for investigation in conscious animals and in full compliance with the central Israeli animal care commission.

In the examples below, if an abbreviation is not defined, it has its generally accepted meaning.

### General Synthetic Methods

### Compound Characterization

Final compounds were confirmed by HPLC/MS (high-performance liquid chromatography/mass spectrometry) analysis and determined to be ≥90% pure by weight. ¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d₆ (residual internal standard CD₃COCD₂H=δ 2.05). The chemical shifts (6) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

### Biological Studies

The biological activities of cannabinoid acid ester compounds of interest such as CBDA-Me are assessed according to a variety of methods, e.g., as described in the examples below.

### Example 1—Synthesis of Cannabinoid Acid (CBDA)

The preparation process described in the PCT application WO 2018/235079 was applied. A mixture of Cannabidiol (CBD, 314 mg, 1 mmol) and 2 molar solution of Magnesium Methyl Carbonate (MMC/2M, 1.5 ml, 3 mmol) in dimethylformamide (DMF) was heated at 130° C. for 3 hours. Then the reaction was cooled to 0° C., acidified with 10% hydrochloric acid and extracted with ether. The organic layer was washed with saline, dried over the drying agent magnesium sulfate (MgSO₄) and then evaporated. The crude compound was then cleaned by column chromatography (20% ether-petroleum ether) to isolate the title compound.

The synthetic methods above are adapted to prepare a variety of cannabinoid acid compounds of interest.

### Example 2—Synthesis of Cannabinoid Acid Methyl Ester (CBDA-Me)

The preparation process described in the PCT application WO 2018/235079 was applied. To a solution of Cannabidiolic Acid (CBDA) (175 mg, 0.488 mmol) in 2.5 ml dichloromethane (CH₂Cl₂), was added 0.02 ml of methanol (CH₃OH, 0.488 mmol) and 7.2 mg of 4-Pyrrolidinopyridine (0.048 mmol). The reaction was stirred for 5 minutes at room temperature followed by the addition of the coupling agent, N,N′ Dicyclohexylcarbodiimide (DCC) (121 mg, 0.585 mmol) and stirred overnight. Then the solvent was evaporated and the crude mixture acidified with 5% hydrochloric acid and extracted with dichloromethane (CH₂Cl₂). The organic layer was washed with saturated aqueous sodium bicarbonate (NaHCO₃), dried over the drying agent magnesium sulfate (MgSO₄) and then evaporated. The crude compound is then cleaned by column chromatography (2% ether-petroleum ether).

The synthetic methods above are adapted to prepare a variety of cannabinoid acid ester compounds of interest.

### Example 3—Synthesis of Tetrahydrocannabinolic Acid Methyl Ester (THCA-Me) from Cannabidiolic Acid Methyl Ester (CBDA-Me)

A preparation process similar to that described in the PCT application publication WO 2002/070506 was applied. BF₃Et₂O (50 μl) is added, under nitrogen atmosphere, to ice cold solution of CBDA (355 mg) in dry methylene chloride (15 ml). The solution is stirred at 0° C. for 1 hour. Saturated aqueous solution of NaHCO₃ (2 ml) is added. The organic layer is removed, washed with water, dried over MgSO₄ and evaporated. The oil is chromatographed on silica gel column (20 g) and eluted with petroleum ether followed by graded mixtures of ether in petroleum ether to isolate the THCA methyl ester.

### Example 4—Synthesis of Tetrahydrocannabinolic Acid (THCA)

A mixture of tetrahydrocannabinol (THC, 314 mg, 1 mmol) and 2 molar solution of Magnesium Methyl Carbonate (MMC/2M, 1.5 ml, 3 mmol) in dimethylformamide (DMF) is heated at 130° C. for 3 hours. Then the reaction is cooled to 0° C., acidified with 10% hydrochloric acid and extracted with ether. The organic layer is washed with saline, dried over the drying agent magnesium sulfate (MgSO₄) and then evaporated. The crude compound is then cleaned by column chromatography (20% ether-petroleum ether) to isolate the title compound.

### Example 5—Synthesis of Tetrahydrocannabinolic Acid Methyl Ester (THCA-Me) from Tetrahydrocannabinolic Acid (THCA)

To a solution of tetrahydrocannabinolic acid (THCA) (175 mg, 0.488 mmol) in 2.5 ml dichloromethane (CH₂Cl₂), is added 0.02 ml of methanol (CH₃OH, 0.488 mmol) and 7.2 mg of 4-Pyrrolidinopyridine (0.048 mmol). The reaction is stirred for 5 minutes at room temperature followed by the addition of the coupling agent, N,N′ Dicyclohexylcarbodiimide (DCC) (121 mg, 0.585 mmol) and stirred overnight. Then the solvent was evaporated and the crude mixture acidified with 5% hydrochloric acid and extracted with dichloromethane (CH₂Cl₂). The organic layer is washed with saturated aqueous sodium bicarbonate (NaHCO₃), dried over the drying agent magnesium sulfate (MgSO₄) and then evaporated. The crude compound is then cleaned by column chromatography (2% ether-petroleum ether) to isolate the title compound.

### Example 6—Synthesis of Cannabierolic Acid (CBGA)

A mixture of cannabigerol (CBG, 316 mg, 1 mmol) and 2 molar solution of Magnesium Methyl Carbonate (MMC/2M, 1.5 ml, 3 mmol) in dimethylformamide (DMF) is heated at 130° C. for 3 hours. Then the reaction is cooled to 0° C., acidified with 10% hydrochloric acid and extracted with ether. The organic layer is washed with saline, dried over the drying agent magnesium sulfate (MgSO₄) and then evaporated. The crude compound is then cleaned by column chromatography (20% ether-petroleum ether) to isolate the title compound.

### Example 7—Synthesis of Cannabierolic Acid Methyl Ester (CBGA-Me) from Cannabiuerolic Acid (CBGA)

To a solution of cannabigerolic acid (CBGA) (180 mg, 0.5 mmol) in 2.5 ml dichloromethane (CH₂Cl₂), is added 0.02 ml of methanol (CH₃OH, 0.5 mmol) and 7.2 mg of 4-Pyrrolidinopyridine (0.048 mmol). The reaction is stirred for 5 minutes at room temperature followed by the addition of the coupling agent, N,N′ Dicyclohexylcarbodiimide (DCC) (121 mg, 0.585 mmol) and stirred overnight. Then the solvent is evaporated and the crude mixture acidified with 5% hydrochloric acid and extracted with dichloromethane (CH₂Cl₂). The organic layer is washed with saturated aqueous sodium bicarbonate (NaHCO₃), dried over the drying agent magnesium sulfate (MgSO₄) and then evaporated. The crude compound is then cleaned by column chromatography (2% ether-petroleum ether) to isolate the title compound.

### Example 8—Synthesis of Cannabinolic Acid (CBNA)

A mixture of cannabinol (CBN, 310 mg, 1 mmol) and 2 molar solution of Magnesium Methyl Carbonate (MMC/2M, 1.5 ml, 3 mmol) in dimethylformamide (DMF) is heated at 130° C. for 3 hours. Then the reaction is cooled to 0° C., acidified with 10% hydrochloric acid and extracted with ether. The organic layer is washed with saline, dried over the drying agent magnesium sulfate (MgSO₄) and then evaporated. The crude compound is then cleaned by column chromatography (20% ether-petroleum ether) to isolate the title compound.

### Example 9—Synthesis of Cannabinolic Acid Methyl Ester (CBNA-Me) from Cannabinolic Acid (CBNA)

To a solution of cannabinolic acid (CBNA) (177 mg, 0.5 mmol) in 2.5 ml dichloromethane (CH₂Cl₂), is added 0.02 ml of methanol (CH₃OH, 0.5 mmol) and 7.2 mg of 4-Pyrrolidinopyridine (0.048 mmol). The reaction is stirred for 5 minutes at room temperature followed by the addition of the coupling agent, N,N′ Dicyclohexylcarbodiimide (DCC) (121 mg, 0.585 mmol) and stirred overnight. Then the solvent is evaporated and the crude mixture acidified with 5% hydrochloric acid and extracted with dichloromethane (CH₂Cl₂). The organic layer is washed with saturated aqueous sodium bicarbonate (NaHCO₃), dried over the drying agent magnesium sulfate (MgSO₄) and then evaporated. The crude compound is then cleaned by column chromatography (2% ether-petroleum ether) to isolate the title compound.

### Example 10—In-Vivo Model of Pain

The therapeutic effect of either THCA-Me, CBGA-Me, CBNA-Me, or a combination thereof on pain is examined in the experimental model of hot-plate test, measuring the analgesic activities of either THCA-Me, CBGA-Me, CBNA-Me, or a combination thereof based on the reaction time of mice to lick their forepaws and/or jump after being placed on an aluminum hot plate heated to, and maintained at about 55° C. as described in the U.S. Pat. No. 5,338,753, with minor modifications. An aluminum surface is maintained at about 55° C. by circulating water through the passages in the metal. A clear plastic cylinder is placed on the surface to prevent escape. The end point is reached when the mouse either performed a hind paw lick or jumped off the surface; in no case are the animals kept more than 30 seconds on the plate. Control values are measured 3 hours before the test values. Mice are treated with THCA-Me, CBGA-Me, CBNA-Me, or a combination thereof at different doses ninety (90) minutes before the hot plate test. The percent change in response time (latency) is calculated by comparing the mean of the control values with the mean of the test values and statistical significance determined by a paired t test.

### Example 11—In-Vivo Model of Inflammatory

The therapeutic effect of either THCA-Me, CBGA-Me, CBNA-Me, or a combination thereof on inflammatory is examined in the experimental model of inflammation (paw edema) in arachidonic acid or platelet activating factor (PAF) injected mice, as described in the U.S. Pat. No. 5,338,753. Briefly, PAF (1.0 μg) or arachidonic acid (1.0 mg) dissolved in 50 μL of 5% ethanol in saline, is injected subcutaneously into the plantar surface of the right hind paw of ether-anesthetized CD-1 female mice (20-25 g). The volume of the right foot is measured to the level of the lateral malleous by water displacement before treatment, fifteen minutes after PAF injection, or thirty minutes after arachidonic acid injection. The mice are then treated with injections of different doses of either THCA-Me, CBGA-Me, CBNA-Me, or a combination thereof. The change in paw volume is calculated for each mouse and the significance for each group is determined by a paired t test.

### Example 12—In-vivo Pentylenetetrazole-induced (PTZ) Model of Generalized Seizures

The antiepileptic effect of either THCA-Me, CBGA-Me, CBNA-Me, or a combination thereof is tested in an acute experimental epilepsy model in pentylenetetrazole (PTZ) injected rats as described in Obay et. al. (Peptides, 2007, 28, 1214-1219). Briefly, adult male Wistar albino rats are divided into a control group, and four experimental groups with seven rats in each group. In order to generate epileptic seizures, PTZ (50 mg/kg) is injected intraperitoneally. The experimental groups receive intraperitoneal injections of either THCA-Me, CBGA-Me, CBNA-Me, or a combination thereof at different doses 30 min before PTZ injection. After PTZ injection, the rats are kept in cages and their behaviors are observed for 30 min. The latencies are separated into three components: first myoclonic jerk (FMJ), generalized clonic seizures (GCS) and tonic generalized extension (TGE).

### Example 13—Cosmetic Formulation for Use as Lip Balm

### General Procedure

In a first beaker, a mixture of beeswax, lanolin and carnauba wax is heated and mixed to 65° C. and then is cooled to around 35° C. In the second beaker, a mixture of cocoa butter, seed oil, tocopherol, and either THCA-Me, CBGA-Me, CBNA-Me, or a combination thereof is heated and mixed to 40° C., then the mixture is poured to the first beaker, and then fragrance oil is added and mixed into the mixture. Finally, the resulted mixture is poured into cylindrical molds and is cooled down to room temperature. The percentage of either THCA-Me, CBGA-Me, CBNA-Me, or a combination thereof within the mixture is about 3% (wt/wt).

An exemplary formulation comprising THCA-Me is shown in Table 1 below.

TABLE 1
Cosmetic Formulation for Use as Lip Balm
Comprising THCA-Me

| Ingredient | Percentage (wt/wt/) |
|---|---|
| Beeswax | 21.00 |
| Lanolin | 18.00 |
| Carnauba wax | 22.00 |
| Cocoa butter | 25.00 |
| seed oil | 5.00 |
| THCA-Me | 3.00 |
| Tocopherol | 1.00 |
| Fragrance agent | 5.00 |
| Total | 100.00 |

An exemplary formulation comprising CBGA-Me is shown in Table 2 below.

TABLE 2
Cosmetic Formulation for Use as Lip Balm
Comprising THCA-Me

| Ingredient | Percentage (wt/wt/) |
|---|---|
| Beeswax | 21.00 |
| Lanolin | 18.00 |
| Carnauba wax | 22.00 |
| Cocoa butter | 25.00 |
| seed oil | 5.00 |
| CBGA-Me | 3.00 |
| Tocopherol | 1.00 |
| Fragrance agent | 5.00 |
| Total | 100.00 |

An exemplary formulation comprising CBNA-Me is shown in Table 3 below.

TABLE 3
Cosmetic Formulation for Use as Lip Balm
Comprising THCA-Me

| Ingredient | Percentage (wt/wt/) |
|---|---|
| Beeswax | 21.00 |
| Lanolin | 18.00 |
| Carnauba wax | 22.00 |
| Cocoa butter | 25.00 |
| seed oil | 5.00 |
| CBNA-Me | 3.00 |
| Tocopherol | 1.00 |
| Fragrance agent | 5.00 |
| Total | 100.00 |

### Example 14—Powdered Edible Additive

A powdered additive is formulated by blending filler such as tapioca maltodextrin or microcrystalline cellulose or a mixture thereof, emulsifier such as soy lecithin granules, flavoring agent, and either THCA-Me, CBGA-Me, CBNA-Me, or a combination thereof. All ingredients are added in a high-speed shearing device at room temperature.

### Example 15—Formulations of Drinking-Water Additive

A drinking-water additive is formulated by forming liposomes that are loaded with either THCA-Me, CBGA-Me, CBNA-Me, or a combination thereof, and then the liposomes are dispersed in water. Optionally an approved food acceptable organic solvent such as ethanol or PEG is added.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are herein incorporated by reference in their entirety, for all purposes.

1. A pharmaceutical composition comprising:
a tetrahydrocannabinolic acid (THCA) ester compound of formula (I): 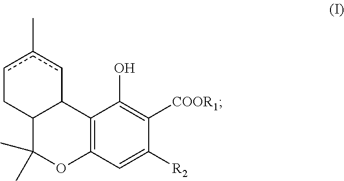
a cannabigerolic acid (CBGA) ester compound of formula (II): 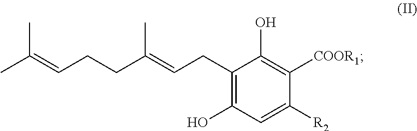
a cannabinolic acid (CBNA) ester compound of formula (III): 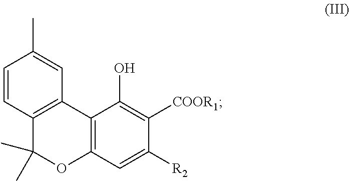
or
a combination thereof;
wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof; and
a pharmaceutically acceptable excipient.

2. (canceled)

3. The pharmaceutical composition of claim 1, wherein R₁ is selected from methyl, ethyl, propyl, butyl, and pentyl.

4. (canceled)

5. (canceled)

6. The pharmaceutical composition of claim 1, wherein R₂ is C₂₋₆ alkyl.

7. (canceled)

8. The pharmaceutical composition of claim 1, comprising a THCA ester compound of formula (Ia): 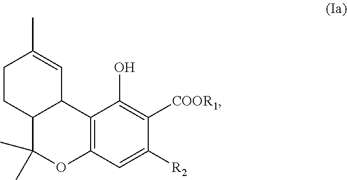
or a pharmaceutically acceptable salt thereof.

9. The pharmaceutical composition of claim 1, comprising a THCA ester compound of formula (Ib): 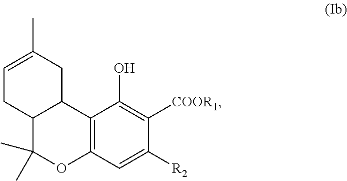
or a pharmaceutically acceptable salt thereof.

10. The pharmaceutical composition of claim 1, comprising Δ⁹-tetrahydrocannabinolic acid methyl ester (Δ⁹-THCA-Me) compound of formula (Ic): 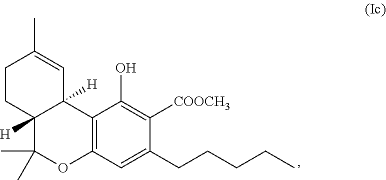 [SMILES: CCCCCc1cc2c(c(O)c1C(=O)OC)[C@@H]1C=C(C)CC[C@H]1C(C)(C)O2]
or a pharmaceutically acceptable salt thereof.

11. The pharmaceutical composition of claim 1, comprising Δ⁸-tetrahydrocannabinolic acid methyl ester (Δ⁸-THCA-Me) compound of formula (Id): 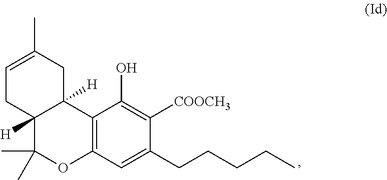 [SMILES: CCCCCc1cc2c(c(O)c1C(=O)OC)[C@@H]1CC(C)=CC[C@H]1C(C)(C)O2]
or a pharmaceutically acceptable salt thereof.

12. The pharmaceutical composition of claim 1, comprising the CBGA ester compound of formula (II).

13. The pharmaceutical composition of claim 1, comprising the CBNA ester compound of formula (III).

14. The pharmaceutical composition of claim 1, further comprising one or more additional active pharmaceutical ingredient (API).

15. The pharmaceutical composition of claim 14, wherein the one or more additional API comprises one or more additional cannabinoid compound.

16.-30. (canceled)

31. The pharmaceutical composition of claim 10, wherein the pharmaceutical composition comprises:
1% to 10% Δ⁹-THCA-Me;
20% to 60% alcohol; and
0% to 35% propylene glycol.

32.-34. (canceled)

35. The pharmaceutical composition of claim 11, wherein the composition comprises:
1% to 10% Δ⁸-THCA-Me;
20% to 60% alcohol; and
0% to 35% propylene glycol.

36.-39. (canceled)

40. The pharmaceutical composition of claim 12, wherein the CBGA ester compound of formula (II) is the CBGA-Me compound of formula (IIa)

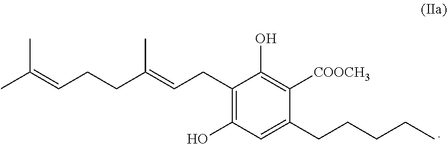 [SMILES: CCCCCc1cc(O)c(C/C=C(\C)CCC=C(C)C)c(O)c1C(=O)OC]

41. The pharmaceutical composition of claim 40, wherein the pharmaceutical composition comprises:
1% to 10% CBGA-Me;
20% to 60% alcohol; and
0% to 35% propylene glycol.

42.-45. (canceled)

46. The pharmaceutical composition of claim 13, wherein the CBNA ester compound of formula (III) is the CBNA-Me compound of formula (IIIa)

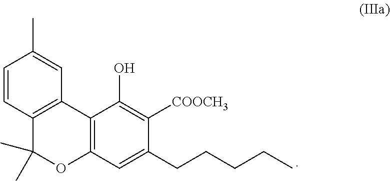 [SMILES: CCCCCc1cc2c(c(O)c1C(=O)OC)-c1cc(C)ccc1C(C)(C)O2]

47. The pharmaceutical composition of claim 46, wherein the pharmaceutical composition comprises:
1% to 10% CBNA-Me;
20% to 60% alcohol; and
0% to 35% propylene glycol.

48.-80. (canceled)

81. A cosmetic or edible composition comprising:
a tetrahydrocannabinolic acid (THCA) ester compound of formula (I): 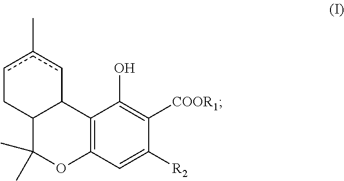
a cannabigerolic acid (CBGA) ester compound of formula (II): 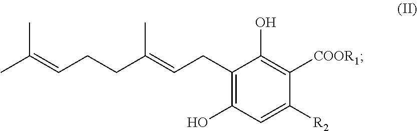
a cannabinolic acid (CBNA) ester compound of formula (III): 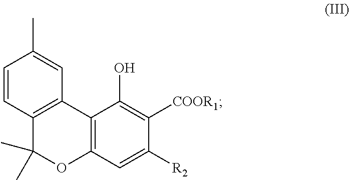
or
a combination thereof;
wherein R₁ and R₂ are independently selected from C₁-C₁₀ alkyl, substituted C₁-C₁₀ alkyl, C₂-C₁₀ alkenyl, substituted C₂-C₁₀ alkenyl, C₂-C₁₀ alkynyl, and substituted C₂-C₁₀ alkynyl, or a pharmaceutically acceptable salt thereof; and
a physiologically acceptable carrier, excipient, or diluent.

82.-158. (canceled)

159. A method of treating, alleviating, or attenuating a disease or disorder in a subject, comprising administering a therapeutically effective amount of a pharmaceutical composition according to claim 1 to the subject, wherein the disease or disorder is selected from the group consisting of pain, impaired neurological function, inflammation, nausea, vomiting, convulsions, psychiatric disorders, low appetite, or glaucoma.

160.-179. (canceled)

180. A method of improving the appearance, the well-being, and/or a health condition in a subject, comprising administering to a subject a cosmetic or edible composition according to claim 81.

181.-187. (canceled)
